(19) United States
(12) Patent Application Publication
(10) Pub. No.: US 2003/0118983 A1
(43) Pub. Date: Jun. 26, 2003

(54) Rapid screening procedure for inflammation mediators

(76) Inventors: Richard A. Cassidy, San Antonio, TX (US); David G. Burleson, San Antonio, TX (US); Marvin L. Salin, Alexandria, VA (US); Angel V. Delgado, San Antonio, TX (US)

Correspondence Address: OFFICE OF THE STAFF JUDGE ADVOCATE, U.S. ARMY MEDICAL RESEARCH AND MATERIEL COMMAND, ATTN: MCMR-JA (MS. ELIZABETH ARWINE), 504 SCOTT STREET, FORT DETRICK, MD, 21702-5012, US

(21) Appl. No.: 10/237,112

(22) Filed: Sep. 9, 2002

Related U.S. Application Data
(63) Continuation-in-part of application No. PCT/US01/07434, filed on Mar. 9, 2001.
(60) Provisional application No. 60/272,048, filed on Mar. 1, 2001.
(60) Provisional application No. 60/188,001, filed on Mar. 9, 2000.

(51) Int. Cl. C12Q 1/00; G01N 33/567

(52) U.S. Cl. 435/4; 435/7.21

(57) ABSTRACT

The invention is compositions and methods for determining the presence of one or more immune response mediators, comprising obtaining a solution containing polymorphonuclear neutrophils, exposing the neutrophils to a chromophore, allowing the chromophore to oxidize to form a luminescent compound, and measuring the level of visible or ultraviolet radiation.

### (B) CROSS-REFERENCE TO RELATED APPLICATIONS

This application is a continuation-in-part of PCT/US01/07434, filed Mar. 9, 2001, which claims priority from U.S. Serial No. 60/272,048, filed Mar. 1, 2001 and U.S. Serial No. 60/188,001, filed Mar. 9, 2000.

### (C) STATEMENT REGARDING FEDERALLY SPONSORED RESEARCH

Not applicable.

### (D) BACKGROUND OF THE INVENTION

(D1) Field of the Invention

The present invention is directed to a screening procedure and compositions for rapidly evaluating drugs that mediate inflammation.

(D2) Description of Related Art

Severe tissue injury, rhabdomyolysis, and myocardial infarction result in the release of myoglobin (Mb) from muscle into the interstitium and the vasculature. Hemoglobin (Hb) is released from erythrocytes during hemolytic disorders, burns, and storage of blood for transfusion. In humans, elevated levels of Mb or Hb have been reported to be associated with acute renal failure, infections, and recurrent cancers in postoperative patients receiving autologous and/or allogeneic transfused blood. It has been shown that trauma patients have a higher incidence of mortality when transfused with acellular, cross-linked oxyhemoglobin (oxyHb) solutions instead of stored red blood cells (RBCs). These and other inflammation cascade immune responses appear to be mediated by or a function of heme protein toxicity.

The addition of Hb or Mb to experimental models of bacterial peritonitis or glomerulonephritis resulted in decreased survival and a reduction of white blood cell count in the rat. OxyHb added to phorbol 12-myristate 13-acetate (PMA)-stimulated polymorphonuclear neutrophils (PMNs) reduces PMN viability, production of O₂⁻, and bacterial killing capability. OxyHb has also been reported to augment the production of nitric oxide (NO) in IL-1-induced cultured smooth muscle cells. Decreasing NO levels by selectively inhibiting the inducible nitric oxide synthase (iNOS) prolongs survival time in the bacterial peritonitis model.

Decreased survival in these infection models induced by heme proteins may be due to a reduced scavenging of NO. by O₂⁻, increasing NO. extracellular levels that damage neighboring cells.

The ability of human PMNs to produce NO. has been controversial. Recently, Wallerath et al. (Thrombosis Haemostasis 77, 163-167 (1997)) have demonstrated neuronal constitutive NOS in PMNs and inducible NOS in eosinophils. Several methods of determining NO. including: NMMA-inhibitable DCFH oxidation, NO-dependent oxidation of oxyHb to metHb and formation of nitrite have reported NO production by PMNs only in the presence of SOD or inhibitors of O₂.⁻ synthesis. Depending on the type and level of PMN stimulation, O₂.⁻ synthesis was 2-50 times NO. levels in these studies. Since activated human PMNs produce both NO and O₂.⁻ which reacts at the rate of diffusion to produce ONOO—, it follows that NO. levels will increase as O₂.⁻ levels are attenuated.

Bass et al. J. Immunol. 130, 1910-1917 (1983)) established the use of DCFH-DA to measure H₂O₂ production in human PMNs by cytofluorometry. Using this procedure, others reported that inhibition of O₂.⁻ production increased DCFH oxidation (i.e., increased DCF fluorescence) in PMNs instead of the expected decrease. Their data indicated that the increased DCFH oxidation was the result of reduced scavenging of NO levels by O₂.⁻ and that this increased DCF fluorescence could be attenuated by NMMA. Recently, this strategy of using L-NMMA or N-nitro-L-arginine to inhibit NO-induced DCF fluorescence was employed in cytofluorometric assessments of NO. levels in neurons and macrophages. In one of these studies, high levels of oxyHb (650 μg/mL) completely inhibited NO.-induced DCF fluorescence, consistent with our findings. In our application of DCFH-DA in measuring intracellular oxidants, DEANO-derived NO. and H₂O₂ were equally potent oxidants, with equal molar concentrations (10⁻¹¹-10⁻⁵ M) inducing similar levels of DCF fluorescence. Since both NO. and H₂O₂ permeate cell membranes, they appear to oxidize DCFH to a similar extent. Although one mole of DEANO can theoretically release 2 moles of NO with a half life of 2.1 min in biological assays, NO. derived from DEANO has been shown to be closer to an equal molar ratio. Higher concentrations of NO. and H₂O₂ (10⁻⁴ M) resulted in reduced levels of DCF fluorescence that could be explained by leakage of DCF through damaged PMN membranes. Currently, the extent to which intracellular DCFH is directly oxidized by NO and/or by the NO. oxidation products, nitrogen dioxide (.NO²) or ONOO⁻, is unresolved.

DCFH oxidation increased linearly in PMA-activated PMNs as the number (i.e., density) of PMNs increased from 50,000 to 500,000 in a fixed volume. Since the mean cellular DCFH oxidation is determined using a constant number of PMNs (i.e.10,000), this indicates that oxidants are originating from neighboring PMNs and that intracellular oxidant load increases with PMN density. Bass et al. also observed an increase in intercellular DCFH oxidation in PMNs from chronic granulomatous disease patients as the percentage of PMA-activated PMNs from normal donors increased. PMNs from chronic granulomatous disease patients activated with PMA do not oxidize DCFH.

This effort stems from the effect of endocrine disruptors like DES (toxahene) with well established effects on cancer causation, reproductive effects, immune disturbances, neurological and neurobehavorial effects. However, no studies have addressed the potential adverse consequences of endocrine disruptors in the pathophysiology of severe trauma or other diseases, where the hypermaetabolic state causes the mobilization of body fat from adipose tissue. Many, if not most endocrine disruptors are lipophilic and are sequestered in lipid depots. Adipose tissues of human were reported to be 10 times greater than that of liver and 100 times greater than that in the brain, kidney or gonadal tissue.

### (E) SUMMARY OF THE INVENTION

NO. has been shown to act as an early proinflammatory mediator by increasing inflammatory transcription factors and cytokine expression Hierholzer et al., J. Exp. Med., 187:917-928 (1998).

Heme protein augmentation of NO. levels may occur in cells that produce both NO. and O₂.⁻. Nitric oxide synthase (NOS) has been found in neutrophils, endothelial cells, macrophages, hepatocytes, Kupffer cells, smooth muscle cells, fibroblasts, and renal epithelial cells. NADPH oxidase, producing varying levels of O₂.⁻, has been characterized in these same cells. In accordance with the present invention, activated PMNs heme proteins bind to the membrane, internalize, and dismute O₂.⁻ to H₂O₂ in a SOD-like manner. This decreases extracellular O₂.⁻, allowing NO. concentrations to rise, enter neighboring cells, and produce toxic effects.

In summary, disease processes that release heme proteins may result in the binding of heme protein to activated PMNs and cause NO.-induced damage to tissue at proximal sites. The amount of tissue damage will be contingent on the type and concentration of oxidants interacting with heme proteins and the levels of antioxidants in the tissue. The described mechanism of the cytotoxic effects of heme proteins suggests that therapeutic interventions should focus on strategies of both reducing NO. levels in tissues and levels of free heme proteins. The first strategy could focus on inhibiting PMNs migration into damaged tissues, inhibiting NO. production, and maintaining adequate antioxidants in the tissues to scavenge oxidants. The second strategy could focus on minimizing damage to cells that release heme proteins, removing heme proteins from serum by plasmapheresis and binding to haptoglobin, reduce binding of heme proteins to cells that produce oxidants, and oxidizing the heme protein to bilirubin by inducing the synthesis of heme oxygenase. Furthermore, our results indicate the potential adverse effects of using cell free oxyHb substitutes (e.g., α-α diaspirin cross-linked Hb) in patients with an activated immune system. This mechanism may explain the increased mortality in trauma patients transfused with crossed-linked Hb.

Finally, our studies may explain the increase in severity of risks related to duration of blood storage and provide support for the study of improved RBC preservation used to treat patients with an activated immune system.

Endocrine disruptors can block the 17-β E₂ beneficial effects by inhibiting interaction with its receptors or by inducing steroid hydroxylases that convert 17-β E₂ to inactive metabolites. Two classes of organochlorine insecticides, cyclodienes (chlordane and heptachlor) and DDT are known to interact with the estrogen receptor. These insecticides markedly reduce serum levels of 17-β E₂ and progesterone in treated rats, alter the transcription of estradiol-regulated genes, eliminate 17-β E₂-induced increases in uterine weight in rodents, as well as altered testosterone, body weight and sexually dimorphoric behaviors in rats at levels found in the US populace.

This study suggests that the potential adverse effects of the estrogenic insecticides cycodienes could be most prominent during the hypermetabolic/inflammatory response to severe injury. During the hypermetabolic response, insecticides as well as other lipophilic chemicals would be released from fat depots. Once released from fat, the availability of these once sequestered xenoestrogens to bind to receptors would increase as serum levels of lipids (e.g. cholesterol) are decreased. Furthermore, there appears to be a synergism among at least one of these xenoestrogens, HE, and an inflammatory cytokine, TNFα also released following injury, resulting in increased NO production and cellular damage in isolated leukocytes. The pathophysiological response to injury activates both metabolic and immune pathways that appears to interact with body burdens of xenoestrogens resulting in the potential for increased levels of morbidity and mortality.

HE, OC, and DDE are lipophilic xenoestrogens that increased with the levels of cholesterol and triglyceride in serum and with age in our subjects. These insecticides accumulate in human adipose tissue, reaching levels 200-500 times those reported in serum, 100 times those in the brain, kidney or gonadal tissue, and 10 times those in hepatic tissue. Following fasting or severe food restriction these xenoestrogen are released from fat stores and increase in serum. Following trauma, increasing levels of TNFα as well as other mediators, increase lipolysis and fat utilization. In burn patients, TNFα levels increase with the severity of the burns, peaking on PBD 3-5, and are higher in nonsurviving patients. Increasing levels of these xenoestrogens could inhibit17β-E₂ dampening effect on lipolysis, resulting in positive feed back loops. Whether the increased levels of HE and OC in non-surviving as compared to surviving patients were due solely to increased lipolysis of fat stores containing equivalent levels of HE and OC with coincident increased fat utilization, or to lipolysis of fat containing elevated insecticide concentrations is unknown. However, since the known major factors accounting for mortality (burn size, inhalation injury, and age) and for transporting xenoestrogensin in serum (cholesterol and triglycerides) were similar in surviving and non-surviving patients, lends support to the possibility of a causal relationship between HE and OC serum levels and mortality.

Further, this model was used to compare the dampening effects of 10 equimolar concentrations (10⁻⁹ to 10⁻⁵ M) of 17β-estradiol (E₂), cortisol, progesterone (P₄), and a combination of E₂ and P₄ after 1 and 5 h on intracellular oxidants. A “two-hit model” of activation, human cytokines (TNF-α, IL1-β, and IFN-γ) combined with hemoglobin, resulted in a 500-600% increase in oxidants that was synergistically greater than the sum of individual treatments of cytokines and hemoglobin. Pharmacological levels of E₂ and cortisol decreased, in a concentration-dependent manner, oxidant levels in MNCs with E₂dampening oxidants to a greater extent at 1 h. None of the steroids reduced oxidants in PMNs, suggesting that the E₂ effect on MNCs was not due to its antioxidant properties. The addition of P₄to concentrations of E₂ almost eliminated oxidants from 1 h-activated MNC. Since L-NMMA inhibited 50% of the total oxidants, at least part of E₂ dampening effects could be attributed to NO.

Clinical and experimental studies using pharmacological doses of cortisol and progesterone have also been shown to be beneficial after injury (Katler E, Weissmann G: Steroids, aspirin, and inflammation. Inflammation 2:295-307, 1977.). In a prospective, double-blind, randomized study, patients treated with the corticosteroids for septic shock had approximately a 10% rate of mortality as compared to 38% of those patients receiving saline. Furthermore, cortisol increased survival time and reduced injury in a ischemia-reperfusion model following arterial occlusions. Likewise, progesterone (P₄) administered following cerebral artery occlusion or cortical injury reduced neuronal death and neurological deficits.

Administering scavengers of reactive oxygen species or inhibitors of nitric oxide synthase reduced endothelial injury comparable to leukocyte depletion, suggesting that reactive oxygen such as nitric oxide (NO) are major mediators in the inflammatory cascade. NO has been reported to induce the production of inflammatory cytokines that may further induce the migration and activation of leukocytes. Previous studies have suggested that E₂ reduces these inflammatory cytokines by attenuation of induction in NO levels following injury.

We modified a previously described isolated human leukocyte model to mimic extravasated leukocytes that are activated in damaged tissue. To activate the leukocytes we used a ‘two-hit’ model of a cytokine cocktail of human inflammatory cytokines (TNFα, IL1β, and IFNγ) combined with human hemoglobin. To approximate early intervention, leukocytes were exposed to stimulants and steroids simultaneously. We compared the effect of 10 equimolar concentrations of estradiol, cortisol, progesterone and a combination of E₂ and P₄ (10⁻⁹ to 10⁻⁵ M) on intracellular oxidant levels and cell membrane damage in both granulocytes (PMNs) and monocytic cells (MNCs) by flow cytometry 1 and 5 h after stimulation.

The accompanying drawings show illustrative embodiments of the invention from which these and other of the objectives, novel features and advantages will be readily apparent.

### (F) DESCRIPTION OF THE DRAWINGS

FIG. 1 shows concentration-dependent effects of H₂O₂ (□) or DEANO-derived NO (▪) on (A) DCFH oxidation and (B) propidium iodide intracellular staining in PMNs. Data represent means±SEM of four experiments. Control vs. concentrations of H₂O₂ or DEANO, *P<0.05.

FIG. 2 shows the effect of PMN density (PMNs/100 μl buffer) on DCFH oxidation as determined in the presence (▪) or absence (□) of PMA. Data represent means±SEM of three experiments. Preparation of PMNs (50,000) vs. PMNs (150,000-1,000,000), *P<0.05.

FIG. 3 shows concentration-dependent effect of oxyMb on DCFH oxidation in unstimulated (▪), unstimulated+L-NMMA (1), LPS-stimulated (Δ), LPS and TNFα-stimulated (□), LPS and TNFα-stimulated+L-NMMA PMNs (◯). Data represent means±SEM of five to seven experiments. Control vs. concentrations of oxyMb for each treatment condition, *P<0.05; unstimulated or LPS and TNFα-stimulated PMNs with vs. without L-NMMA, #P<0.05.

FIG. 4 shows Concentration-dependent effects of heme proteins, RBCs, or ferrous iron on DCFH oxidation in PMA-stimulated PMNs with (A) and without (B) arginine. (A) Arginine-loaded PMNs were treated with varying concentrations of oxyMb (▪), oxyHb (▴), RBCs (◯), sonicated RBCs (1), bilirubin (Δ [symbol] , or ferrous iron (□). (B) PMNs without arginine were treated with varying concentrations of oxyMb (▪), mixed oxyMb and metMb (□), oxyHb-A₀ (◯), α-α diaspirin cross-linked oxyHb (▴), mixed oxyHb and metHb (1), or metHb (Δ). Data represent means±SEM of four experiments. Control vs. concentrations of heme proteins, RBCs, or iron,*P<0.05; oxyMb vs. comparable oxyHb concentrations, #P<0.05.

FIG. 5 shows concentration-dependent effects of oxyMb+100 U/mL Cu,Zn SOD on DCFH oxidation (▪) and nitrite levels (□) in PMA-stimulated PMNs. Data represent means±SEM of five or seven experiments, respectively. Control vs. concentrations of oxyMb on DCF fluorescence and nitrite levels in filtrates in PMNs, *P<0.05.

FIG. 6 shows oxyHb binding to and internalization into stimulated and unstimulated PMNs. Binding was assessed by using OxyHb-conjugated fluorescein-EX (4.5 molecules/oxyHb). OxyHb-FLEX (□), OxyHb-FLEX+LPS and TNFα (▴) and OxyHb-FLEX+PMA (▪). Data represent means±SEM of four to five experiments. Control vs. concentrations of oxyHb-FLEX bound to and internalized into unstimulated or stimulated PMNs, *P<0.05; OxyHb-FLEX vs. comparable OxyHb-FLEX+PMA concentrations, #P<0.05.

FIG. 7. Bound and internalized OxyHb-FLEX fluorescence in PMA-activated PMNs. (A) Whole cell and (B) Z-plane confocal images of PMNs incubated with OxyHb-FLEX (250 μg/mL).

FIG. 8. Effects of NOS inhibitors, antioxidants, and Cu, Zn SOD on oxyMb-induced DCFH oxidation in PMA-stimulated PMNs. (A) PMNs in the absence (▪) or presence of L-NIO, 5 mM (▴); L-NMMA, 5 mM (◯); glutathione, 25 μM and ascorbate, 25 μM (□) or human erythrocyte Cu,Zn SOD, 30 U (1). Data represent means±SEM of five to seven experiments. Control vs. concentrations of oxyMb, *P<0.05; oxyMb vs. comparable concentrations of oxyMb+L-NMMA, L-NIO, or SOD, #P<0.05; oxyMb+L-NMMA vs. comparable concentrations of oxyMb+glutathione and ascorbate, ⁺P<0.05 or (B) Various concentrations of bovine liver Cu,Zn SOD (30-10,000 U/mL) in the presence oxyMb (30 82 g/mL) with (□) and without L-NMMA (▪). Data represent means±SEM of three experiments. OxyMb only vs. Cu, Zn SOD+oxyMb, *P<0.05; without vs. with L-NMMA, #P<0.05.

FIG. 9. Concentration-dependent effects of oxyMb (▪) or SOD (□) on extracellular O₂.⁻ produced by PMA-stimulated PMNs. Data represent means±SEM of four experiments.

FIG. 10. Concentration-dependent effect of oxyMb on four apoptotic endpoints in PMA-stimulated PMNs. (A) OxyMb effect in the absence (▪) or presence of L-NMMA, 5 mM (□) on the phosphatidylserine externalization on PMNs plasma membrane as determined by annexin V binding. (B) OxyMb effect in the absence (▪) or presence of L-NMMA, 5 mM (□) on DNA strand breaks in PMNs. Labeling solution without terminal transferase (hatched bar) and with DNase I, (cross-hatched bar) were used as negative and positive controls, respectively. (C) OxyMb effect in the absence (▪) or presence of L-NMMA, 5 mM (□) on the viability of PMA-stimulated PMNs as determined by intracellular levels of propidium iodide (PI). (D) OxyMb effect in the absence (▪) or presence of L-NIO, 5 mM (□) on ATP levels in PMA-stimulated PMNs. Data represent means±SEM of three to seven experiments. Control vs. concentrations oxyMb,*P<0.05; oxyMb vs comparable concentrations of oxyMb+L-NMMA, or L-NIO, *P<0.05.

FIG. 11. Effect of density of PMA-stimulated PMNs on co-incubated lymphocyte viability in the absence (▪) or presence of oxyMb (30 ug/mL) (□), or oxyMb (30 μg/mL)+L-NMMA, 5 mM ( ) as determined by intracellular levels of propidium iodide. Data represent means±SEM of six experiments. OxyMb vs. without oxyMb, *P<0.05; oxyMb vs. comparable concentrations of oxyMb+L-NMMA, #P<0.05.

FIG. 12. Effect of HE on PMN-derived NO on cell membrane integrity in neighboring cells. HE reduced cell membrane integrity (P<0.001) in co-incubated lymphocytes (□) at PMN/lymphocytes ratios≧2 as compared to untreated lymphocytes (▪). HE-induced cell membrane damage in surrounding lymphocytes was attenuated by the addition of either L-NMMA ( [symbol] ) (P<0.01) or PTIO (●) (P<0.001). Data represent means±SEM of six experiments.

FIG. 13. Effects of HE and 17β-E2 on PMN-derived NO and on NO-induced DNA strand breaks in PMNs. HE (□) (a) and 17β-E2 (□) (b) increased the intracellular level of NO and HE (c) increased DNA strand breaks in PMNs in a concentration-dependent, biphasic manner. Co-incubation of HE (a, c) or 17β-E2 (b) treated PMNs with L-NMMA ( [symbol] ), tamoxifen (●), or ICI 182,780 (◯) decreased induced levels of NO(□) (a, b);and DNA strand breaks (c). Co-incubation of HE with TNFα (▪) increased HE-induced NO levels (a) and DNA strand breaks (c) and caused a 10-fold left shift in the concentrations of HE that induced peak responses (a, c). Data represent means±SEM of PMNs isolated from 4-6 different donors except tamoxifen in FIGS. 13a & 13b (n=3 & 1 respectively). (**P<0.01) compared to control values.

FIG. 14 compares the oxidant levels in monocyte cells (MNC) and granulocytes (PMN) when unstimulated, after cytokine stimulation, after hemoglobin stimulation, and after combined hemoglobin/cytokine stimulation.

FIG. 15 shows the effect of estradiol on levels of intracellular oxidants in MNCs (FIG. 15A) and PMNs (FIG. 15B).

FIG. 16 shows the effect of progesterone on levels of intracellular oxidants in MNCs (FIG. 16A) and PMNs (FIG. 16B).

FIG. 17 shows the effect of cortisol on levels of intracellular oxidants in MNCs (FIG. 17A) and PMNs (FIG. 17B).

FIG. 18 shows the effect of the combination of progesterone and estradiol on levels of intracellular oxidants in MNCs (FIG. 18A) and PMNs (FIG. 18B).

### (G) DETAILED DESCRIPTION OF THE INVENTION

The present invention is a method for determining the presence of one or more immune response mediators, such as inflammation mediators, comprising obtaining a solution containing polymorphonuclear neutrophils (PMNs), exposing the neutrophils to a chromophore, allowing the chromophore to oxidize to form a luminescent compound, and measuring the level of visible or ultraviolet radiation. In some embodiments of the invention, the method is conducted in vitro. In some embodiments of the invention, the luminescent compound is a fluorescent compound.

In accordance with one aspect of the present invention, the level of radiation corresponds to or is an indicator of the amount of deleterious effect caused by the inflammation mediator.

In accordance with another aspect of the present invention, the level of radiation corresponds to or is an indicator of the amount of counter-agent to the inflammation mediator that may be appropriate to administer to the human or animal that produced the inflammation response.

The approach of using isolated human leukocytes activated with relevant human inflammatory mediators as a model, may provide insights into inflammatory pathways and enable the selection of a drug or combination of drugs that will dampen major processes of the inflammatory cascade. Intracellular oxidant production, expression of adhesion molecules, and expression of cytokines and chemokines in/on leukocytes are major cellular adaptations to injury that amplify the inflammatory cascade. These leukocytes' adaptations can be measured by using cytofluorimetric procedures and can be used as endpoints for selecting treatments that dampen specific inflammatory processes following injury. The selected drug or combination of drugs derived from these cost-effective, proximal in vitro procedures can then be further validated in appropriate animal models.

In preferred embodiments of the invention, the solution containing polymorphonuclear neutrophils comprises a body fluid, including but not limited to blood. In some embodiments of the invention, the solution, such as whole blood, includes PMNs as a natural constituent of the solution. In other embodiments of the invention, PMNs may be added to the solution.

In preferred embodiments of the invention, the chromophore is any molecule, or group or groups in a molecule, which absorb visible or ultraviolet radiation. Exemplary groups include, but are not limited to —CH═CH—, —N═N—, and —C(═O)—. These groups are typically unsaturated groups, and typically produce color when conjugated. The preferred class of chromophores are those effective with flow cytometry protocols, i.e., those that fluoresce at a certain wavelength, such as a fluorochrome. The preferred fluorochrome is a fluorescein compound. In some embodiments of the invention, the fluorescein compound may be unconjugated, preferably comprising a salt of acetic acid. In these embodiments, the preferred fluorescein compound is 2,7-dichlorodihydrofluorescein diacetate (DCFH-DA). In some embodiments of the invention, the fluorescein compound may be conjugated, preferably conjugated to a heme protein or conjugated to a molecule that contains a heme group. An exemplary heme protein includes, but is not limited to hemoglobin. A molecule that includes a heme group includes, but is not limited to myoglobin.

In accordance with the present invention, allowing the chromophore to oxidize comprises contacting the chromophore with intracellular oxidants formed as a function of the inflammation process. Exemplary intracellular oxidants that are by-products and/or markers of the inflammation cascade include, but are not limited to, nitric oxide (NO), hydrogen peroxide (H₂O₂), superoxide (O₂⁻) and peroxynitrite (OONO⁻).

In accordance with the present invention, the production of these oxidants are indicators or markers of immunosuppression, immunomodulation, and/or inflammation modulation; or of the presence of one or more molecules, compounds, groups, or substances that cause or involved in immunosuppression, immunomodulation, and/or inflammation modulation.

In accordance with the present invention, any method of measuring the level of radiation may be used. In preferred embodiments of the invention, the method involves measuring the amount of visible or ultraviolet light generated by an activated chromophore. In most preferred embodiments of the invention, the method involves measuring the amount of any chromophore suitable for use with flow cytometry.

A method of treatment comprising determining the level of inflammation mediators present in a body fluid, and administering an agent counter to the inflammation mediator. In some embodiments of the invention, determining the level of inflammation mediators comprises at least one of exposing PMNs to a chromophore, oxidizing the chromophore to produce luminescence, oxidizing the chromophore to produce fluorescence, measuring the level of luminescence or fluorescence, or combinations thereof.

Another embodiment of the present invention includes the use of hemoglobin-conjugated fluorescein to evaluate endocytic processes that are indicators of inflammation mediation, e.g., inflammatory cytokine receptor interactions and/or nitric oxide production, using a method as described above.

Another embodiment of the present invention includes screening or evaluating drugs that modulate one or more steps in the inflammation cascade, using a method as described above.

Another embodiment of the present invention includes screening or evaluating various concentrations of drugs (response may be concentration dependent), using a method as described above.

Another embodiment of the present invention includes screening or evaluating combination of drugs and/or combinations of concentrations, using a method as described above.

These and other inventions will be evident to those skilled in the art.

Each of these elements will now be described in more detail.

As shown in more detail in the Examples, the compositions and methods of the present invention are suitable for use with an injury, condition, or event that stimulates the immune response, particularly those that involve the formation or production of nitric oxide (NO), hydrogen peroxide (H₂O₂), superoxide (O₂⁻), and peroxynitrite (OONO⁻). Specific examples are described in the Examples, but the invention should not be limited thereby.

As used herein, inflammation refers to an animal's or mammal's, preferably human, localized response by vascularized tissues to injury caused by chemical, physical, or biological agents. Events that follow injury include, but are not limited to vasodilation, stasis, leukocyte margination and emigration, and exudation of leukocytes and plasma. The purpose of inflammation is to dilute, contain, and destroy the injurious agent. As is well known to those skilled in the art, a wide variety of molecules are involved in inflammation processes or the inflammation cascade.

As used herein, an inflammation mediator is any molecule, compound, or substance that increases or decreases inflammation or a portion of the inflammation process, typically in a manner that does not benefit the animal or patient.

As used herein immunomodulation refers to any molecule that raises or lowers an immune response above or below what is normal or within a normal range. Included within the present invention is any immune system response that involves a modulated level of nitric oxide (NO), hydrogen peroxide (H₂O₂), superoxide (O₂⁻), and peroxynitrite (OONO⁻). Specific exemplary immune responses include but are not limited to a wound, a burn, tissue injury, severe tissue injury, rhabdomyolysis, myocardial infarction, hemolytic disorders, biological fluid transfusion, such as a blood or blood product transfusion, autologous and/or allogeneic blood product transfusion, acute renal failure, infections, cancer, chronic or recurrent cancer, heme protein transfusions, bacterial infections, such as peritonitis, kidney disorders, such as glumerulonephritis. Abbreviations used in this specification: PMNs, polymorphonuclear neutrophils; oxyMb, oxymyoglobin; oxyHb, oxyhemoglobin; NO., nitric oxide; O₂.⁻, superoxide; PMA, phorbol myristate acetate; ONOO—, peroxynitrite; DCFH-DA, 2,7-dichlorodihyrofluorescein-diacetate; DCF, 2,7-dichlorofluorescein; LPS, ]lipopolysaccharide; TNFα, tumor necrosis factor α; SOD, superoxide dismutase; oxyHb-FLEX, fluorescein-5-EX succinimidyl ester conjugated to oxyhemoglobin; NOS, nitric oxide synthase; RBC, red blood cell, DEANO, diethylamine nitric oxide; H₂O₂, hydrogen peroxide; L-NMMA, N-methyl-L-arginine; L-NIO, N-(1-iminoethyl)-L-ornithine; PI, propidium iodide; SIN-1, 3-morpholinosynonimine.

### EXAMPLES

### Example 1

### PMNs and Lymphocyte Isolation

All reagents, unless otherwise specified, were obtained from Sigma Chemical Co. (St. Louis, Mo). Human PMNs and lymphocytes were obtained from EDTA-preserved venous blood of non-smoking adult males by layering blood over Polymorphprep, Nycomed Pharma As (Oslo, Norway). Blood was centrifuged at 550 g for 30 min at 22° C., and the PMN and lymphocyte layers were collected and washed twice in Hanks' balanced salt solution (HBSS) Gibco (Grand Island, N.Y.). All PMNs were incubated with physiological levels (10⁻⁴ M) of arginine unless otherwise stated. One or two hypotonic lyses were performed to lower the red blood cells to ≦1% of PMNs or of lymphocytes. RBC ghosts lying on top of the PMNs were removed after the first hypotonic lyse by pipette extraction. The purity of PMNs and lymphocytes was greater than 88% and 95%, respectively, and their viability as determined by trypan blue-uptake was greater than 90%. All experiments were conducted with 300,000 PMNs added to 100 μl of HBSS in tubes (12×75 mm) opened to room air unless otherwise stated. Replicate experiments were conducted using PMNs isolated from different donors.

### Example 2

### Effect of NO. or H₂O₂ Concentration or PMN Density on DCFH Oxidation

PMNs were incubated with 2,7-dichlorodihydrofluorescein diacetate (DCFH-DA; (2 μM; from Kodak, Rochester, N.Y.) at 37° C. for 15 minutes. DCFH-DA permeated the cells freely and was trapped after enzymatic hydrolysis of the diacetate to DCFH. Oxidation of DCFH resulted in the fluorescent DCF. To assess the use of DCFH-DA in measuring intracellular levels of NO and/or H₂O₂, a NO. donor, diethylamine nitric oxide (DEANO; Molecular Probes, Eugene, Oreg.) or hydrogen peroxide (H₂O₂), at concentrations of (10⁻¹¹ to 5×10⁻⁴ M) were incubated with DCFH-loaded PMNs at 37° C. for 30 min. Concentrations of DEANO-derived NO. were generated by rapidly performing an initial 10-fold serial dilution of a concentrated (10⁻² M), alkali (1 mM NaOH) stock into HBSS buffer followed by a 10 fold dilution into the PMN preparations. Intracellular levels of propidium iodide (PI) were used as a measure of cell membrane integrity. PI staining of oxidant-treated PMNs (as described above) was determined by flow cytometry analyses after a 2-3 min treatment with PI (0.75 mM). The effect of density of PMNs on DCFH oxidation was assessed by incubating increasing numbers of PMNs (50,000-1,000,000)/100 μl HBSS) with PMA (200 nM) for 30 min.

Flow cytometric analyses were performed with an argon laser (488 nm) and emission light measured behind a filter transmitting 530/30 nm light on a FACScan, Becton Dickinson (San Jose, Calif.) with CELLQuest data acquisition and analysis software. For each sample, 10,000 PMNs were collected. The mean channel fluorescence was determined on a linear scale from a single parameter histogram. Mean channel fluorescence was presented as molecules of equivalent fluorescein using a standard curve prepared from fluorescein microbeads, Flow Cytometry Standards Corp. (San Juan, PR).

### Example 3

### Heme Protein Effect on DCFH Oxidation in Unstimulated, Lipopolysaccharide (LPS) and Tumor Necrosis Factor α (TNFα)-, and PMA-stimulated PMNs

DCFH-loaded PMNs were incubated with and without N-methyl-L-arginine (L-NMMA; 5 mM; Calbiochem, San Diego, Calif.) and heme proteins (0.3-1000 μg/mL) at 37 ° C. for 10 minutes. The L-NMMA-treated PMNs were subsequently incubated for an additional 30 min in the absence or presence of LPS (10 ng/mL), LPS (10 ng/mL) and TNFα (1 ng/mL), or PMA (200 nM). In unstimulated and LPS and TNFα -stimulated PMNs, the treatments were oxyMb, or oxyMb+L-NMMA (5 mM). In PMA-stimulated PMNs with arginine, the treatments were oxyMb, oxyHb, bilirubin, ferrous chloride or either isolated RBCs or sonicated RBCs containing comparable amounts of oxyHb. RBC cell membranes were ruptured using a micro-ultrasonic cell disrupter, Kontes, Janke and Kukel Gmbh and CO. (Stauten, Germany). In PMA-stimulated PMNs without arginine, the treatments were oxyMb (dithionite-treated), oxyMb, chromatographically-purified oxyHb-A₀ (98% ferrous), α-α diaspirin cross-linked oxyHb (98.6% ferrous), oxyHb, or metHb. OxyMb was prepared by mixing excess sodium dithionite with Mb for 1 h and inorganic sulfur compounds were removed by dialyzing in HBSS buffer for 3 h. Percent iron in the ferric state in non-dithionite treated oxyMb or oxyHb was not assessed. Endotoxin levels in α-α Hb as determined by limulus amebocyte lysate assay was 0.125 EU/mL. Diaspirin cross-linked Hb was a gift from Walter Reed Army Institute of Research.

To assess whether heme proteins quench DCF fluorescence, PMNs were loaded with oxidized DCF (2 uM) Kodak (Rochester, N.Y.) and incubated with varying myoglobin concentrations (1-3,000 ug/mL). Flow cytometric measurements of oxidant-induced DCF fluorescence were performed as described above.

### Example 4

### OxyMb Effects on NO₂⁻ and NO₃⁻ Levels

Total nitrate and nitrite levels in filtrates of PMNs were determined by reducing the nitrate to nitrite by nitrate reductase and reacting the resulting nitrite with 2,3-diaminonaphthalene to form a fluorescent product. PMNs (500,000/300 ul HBSS) were treated with oxyMb (1-1000 82 g/mL) and SOD (30 U) for 10 min followed by PMA (200 nM) for 1 h at 30° C. Filtrates were collected by centrifugation at 16,000 g for 15 min through Ultrafree-MC 10,000 NMWL filters (Millipore, Bedford, Mass.). Total nitrite levels were determined with a 96-well fluorescence analyzer (IDEXX, Westbrook, Me.). PMNs exposed to the same treatment were analyzed by flow cytometry for DCFH oxidation.

### Example 5

### OxyHb Bound to and Internalized into PMNs

OxyHb bound to and internalized into PMNs was determined using fluorescein-conjugated oxyHb (Molecular Probes, Eugene, Oreg.). Each molecule of chromatographically purified oxyHb-A₀ (98% ferrous) had an average of 4.5 molecules of fluorescein-5-EX succinimidyl ester conjugated to lysine (oxyHb-FLEX). OxyHb-FLEX (1-250 μg/mL) was incubated with PMNs with and without albumin (20 mg/mL) for 10 min in a shaking water bath at 37° C. followed by a 30 min incubation with or without LPS (10 ng/mL) and TNFα (1 ng/mL) or PMA (200 nM). Flow cytometric measurements were performed as described above. To visualize intracellular levels of oxyHb-FLEX, whole cell and Z-plane images of PMNs were collected using an Axiovert 135 inverted confocal microscope (Zeiss, Thornwood, N.Y., and Atto Instruments, Rockville, Md.).

### Example 6

### OxyMb Effects on DCFH Oxidation in PMA-activated PMNs With and Without NO inhibitors, Cu, Zn SOD or Antioxidants

DCFH-loaded PMNs were incubated for 10 min at 37° C. with various amounts of oxyMb (1-1000 μg/mL) with or without L-NMMA (5 mM), N-(1-iminoethyl)-L-ornithine (L-NIO) (5 mM), ascorbate (25 μM) and glutathione (25 μM), human albumin (20 mg/mL), or human erythrocyte Cu, Zn SOD (300 U/mL; 80 μg/mL) before a 30 min incubation with PMA (200 nM). Also, various concentrations of bovine liver Cu, Zn SOD (30-10,000 U/mL; 8-2640 μg/mL) and 30 μg/mL oxyMb were incubated with PMA-stimulated PMNs pretreated with and without L-NMMA. Flow cytometric measurements of oxidant-induced DCF fluorescence were performed as described above.

### Example 7

### Comparison of oxyMb and SOD Effect on Extracellular O₂.⁻ Levels

PMNs (200,000/200 μl HBSS) were treated with various concentrations of oxyMb or bovine liver Cu, Zn SOD (0.05-1000 μg/mL) in a 340 ATTC 96-well reader (SLT-Labinstruments, Salzburg, Austria) and activated with PMA. O₂.⁻ production was determined by measuring reduction of cytochrome c, at 550 nm.

### Example 8

### Phosphatidylserine Externalization on PMN's Plasma Membrane

Levels of phosphatidylserine on the outer surface of plasma membrane were determined by FITC-labeled annexin V (8 μg per 300,000 PMNs/100 μL) according to manufacturer's instructions (Caltag Laboratories, Burlingame, Calif.). PMNs were incubated with and without L-NMMA (5 mM) and oxyMb (1-1000 μg/mL) for 10 min at 37° C. followed by PMA (200 nM) for 1 h at 37° C. PMNs were then incubated with annexin V for 30 min and propidium iodide (0.75 mM) for 2-3 min at 4° C. prior to flow cytometric analyses.

### Example 9

### DNA Strand Breaks

OxyMb effect on DNA strand breaks in PMA-activated PMNs was measured by flow cytometry using the tdt-mediated dUTP nick end labeling assay (In Situ Cell Death Detection Kit, Fluorescein, Boehringer Mannheim, Indianapolis, Ind.) according to manufacturer's instructions. PMNs were incubated with and without NMMA (5 mM) and oxyMb (1-1000 μg/mL) for 10 min at 37° C. PMNs were subsequently treated with PMA (200 nM) for 2 h at 37° C. Label solutions without terminal transferase or with DNase I (10 U) served as negative and positive controls, respectively.

### Example 10

### Propidium Iodide (PI) Determination of PMN and Lymphocyte Viability

Intracellular levels of PI were used as a measure of cell membrane integrity. PMNs were incubated with and without L-NMMA (5 mM) and oxyMb (1-1000 μg/mL) for 10 min at 37° C. followed by PMA (200 nM) for 2 h at 37° C. Membrane integrity was determined flow cytometry analyses after a 2-3 min treatment with PI (0.75 mM).

### Example 11

### ATP Levels in PMNs

OxyMb effect on ATP levels in PMA-activated PMNs was measured using the Bioluminescent Somatic Cell Assay Kit according to the manufacturer's instructions. PMNs were incubated with and without L-NIO (5 mM) and oxyMb (1-1000 μg/mL) for 10 min at 37° C. PMNs were subsequently treated with PMA (200 nM) for 2 h at 37° C. PMNs were lysed, frozen at −20° C. and analyzed within 24 h. Emitted light was determined in a camera luminometer, Tropix (Redford, Mass.) using Panchromatic black and white, ISO 20,000, type 612 film, Polaroid (Cambridge, Mass.) with an exposure time of 2.5 min. Level of exposure was measured and digitized with a solid state camera, COHU (San Diego, Calif.) and analyzed using the public domain NIH Image 1.55 program. Statistical analysis. All values are presented as mean±SEM. The Kolmogorov-Smirnov test was used to verify the normal distribution of the data. Groups were compared by using analysis of variance with the Fisher least significant difference (LSD) post hoc procedure. Statistical significance was accepted at P values<0.05.

### Example 12

### DCFH as an Indicator for Measuring Oxidants in PMNs

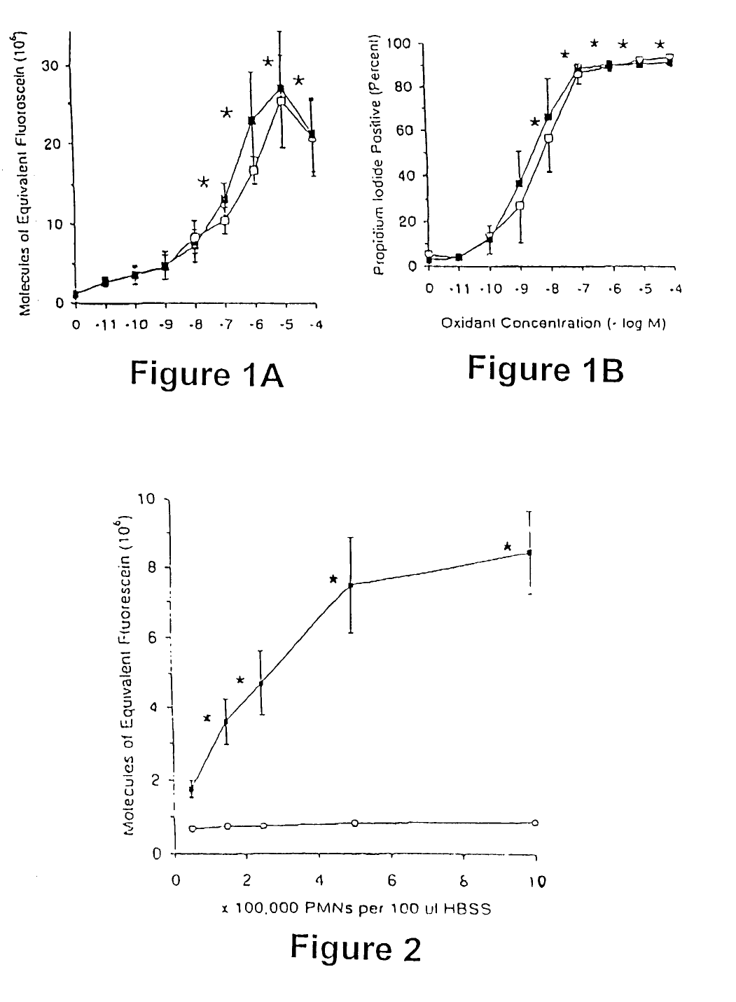

Increasing molar concentrations of DEANO-derived NO. and H₂O₂ (10⁻¹¹-10⁻⁵ M) incrementally increased DCFH oxidation to similar levels, increasing 25-fold over controls levels at 10⁻⁵ M. However, the 10⁻⁴ M concentration of both oxidants resulted in an approximately 25% decrease (FIG. 1A). Both NO. and H₂O₂ also incrementally increased cellular membrane damage with 87% of PMNs staining positive for propidium iodide at levels≧10⁻⁷ M (FIG. 1B). Loss of cellular membrane integrity with leakage of DCF from PMN may account for the loss of fluorescence at the 10⁻⁴ M concentration of oxidants. PMA-activated PMNs showed an approximate 5-fold incremental increase in oxidant-induced DCF fluorescence as the number of PMNs increased from 50,000 to 500,000 in a fixed volume of buffer (FIG. 2).

### Example 13

### Heme Protein Effects on DCFH Oxidation (NO.) and Nitrite Levels

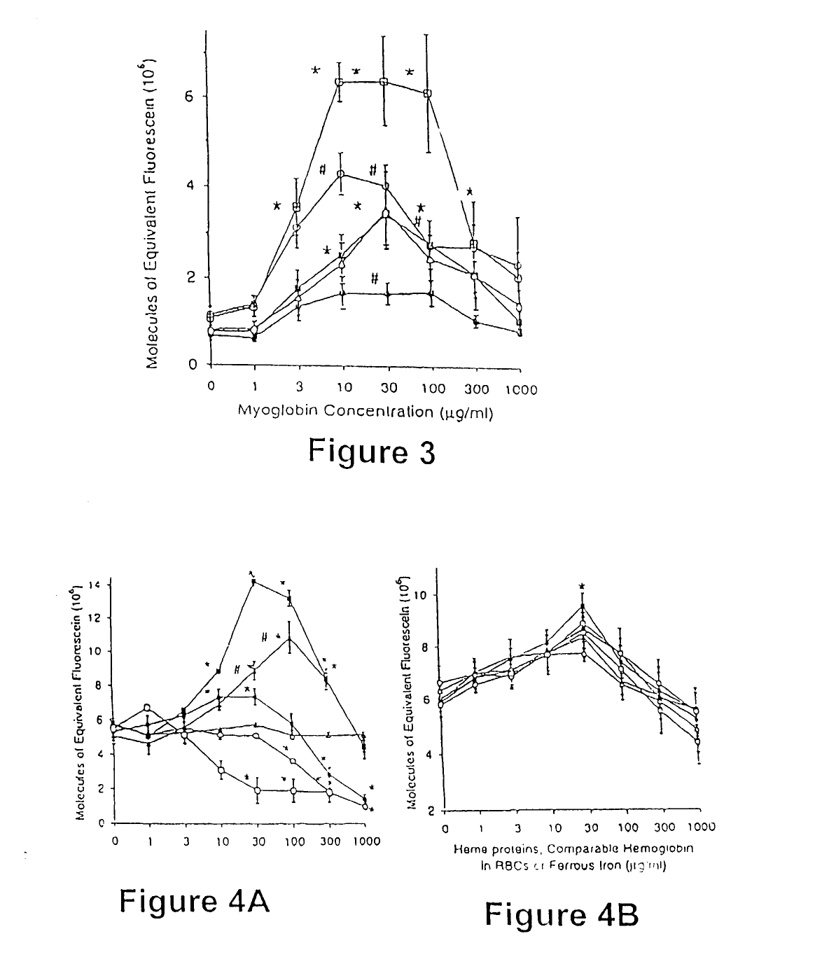

None of the oxyMb concentrations (0-3000 μg/mL) used in this study quenched DCF fluorescence in PMNs (data not shown). OxyMb at concentrations of 1-1000 μg/mL incubated with unstimulated, LPS and TNFα, and PMA-stimulated PMNs induced a concentration-dependent, biphasic response in DCFH oxidation. In unstimulated PMNs, 30 μg/mL of oxyMb induced an approximately 4-fold increase in DCFH oxidation compared with the 0 and 1000 μg/mL oxyMb concentrations (FIG. 3). L-NMMA, a competitive inhibitor of NOS, lowered the oxyMb (30 μg/mL)-induced increase in DCFH oxidation by 50%. In LPS and TNFα-stimulated PMNs, DCFH oxidation peaked at 30 ug/mL of oxyMb with an approximately 6- and 3-fold increase in DCFH oxidation compared, respectively, with the 0 and 1000 μg/mL oxyMb concentrations (FIG. 3). Again, L-NMMA lowered the oxyMb (30 μg/mL)-induced increase in DCFH oxidation by 38%.

In PMA-stimulated PMNs, oxyMb produced a concentration-dependent increase in DCFH oxidation, with the peak response at 30 μg/mL. This level of oxyMb increased DCFH oxidation approximately 3-fold compared with the 0 and 1000 μg/mL oxyMb concentrations (FIG. 4A). Oxidant levels in PMA-stimulated PMNs treated with 0 and 30 μg/mL oxyMb were 6- and 2-fold higher than that of LPS and TNFα-stimulated PMNs, respectively (FIG. 4 vs. FIG. 3). The oxyHb effect on DCFH oxidation paralleled oxyMb except that oxyHb was approximately 20-40% less efficient in inducing DCFH oxidation. RBC preparations containing comparable amounts of oxyHb as above did not increase DCFH oxidation, but did produce an incremental decrease in DCFH oxidation at RBC concentrations of oxyHb≧100 μg/mL. When comparable concentrations of RBCs were sonicated, DCFH oxidation was intermediate between oxyHb and intact RBCs, producing higher DCFH oxidation than intact RBC at 10, 30, and 100 μg/mL and DCFH oxidation comparable to that of intact RBC at 300 and 1000 μg/mL. Thus, the encapsulation of oxyHb in the RBC membranes appears to have reduced the DCFH oxidation levels. Bilirubin, the non-protein breakdown product of hemoglobin, did not affect DCFH oxidation. Addition of FeCl₂.4H₂O produced a decrease in DCFH oxidation above 30 μg/mL. On a molar basis, the Fe²⁺ in 1 μg/mL of FeCl₂.4H₂O equates to 85 μg/mL oxyMb. Iron-induced decreases in DCFH oxidation coincided with decreases in PMN viability (data not shown).

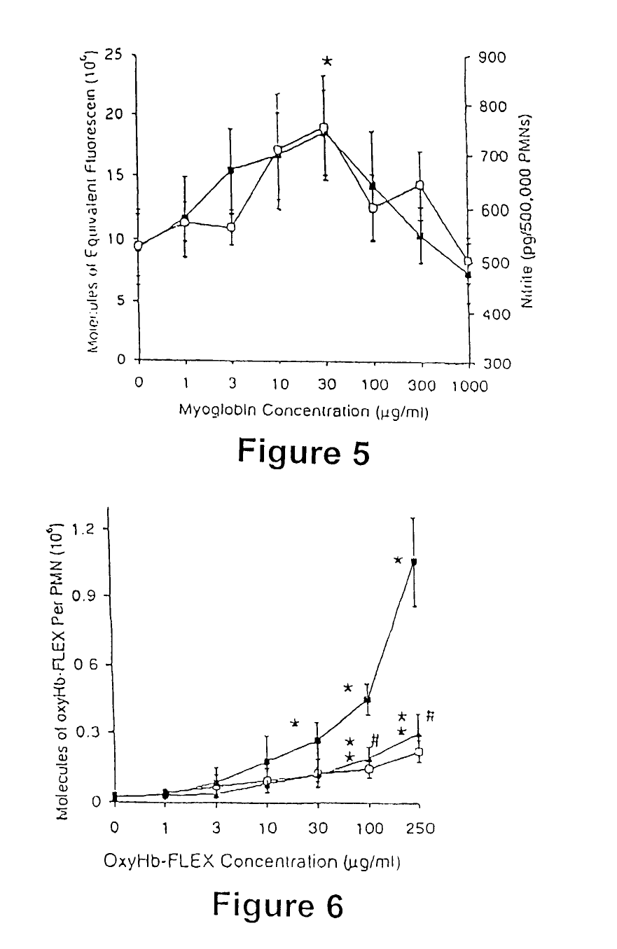

PMA-stimulated PMNs were also treated with oxyMb (pretreated with dithionite), oxyMb, chromatographically-purified oxyHb-A₀ (98% ferrous), α-α diaspirin cross-linked oxyHb (98.6% ferrous), oxyHb, or metHb in HBSS without arginine (FIG. 4B). All heme proteins except metHb produced similar DCFH oxidation profiles, inducing an increase in DCFH oxidation at the 30 μg/mL concentration. Removing arginine from the preparation decreased the heme protein-induced DCFH response by 25-50% (FIGS. 4A vs. 4B). Levels of nitrite, a stable product of oxidized NO, in the PMN filtrates paralleled the profile of DCFH oxidation (FIG. 5). OxyMb (30 μg/mL)-treated PMNs produced a doubling of nitrite levels as compared with untreated PMNs.

### Example 14

### OxyHb Bound to and Internalized into PMNs

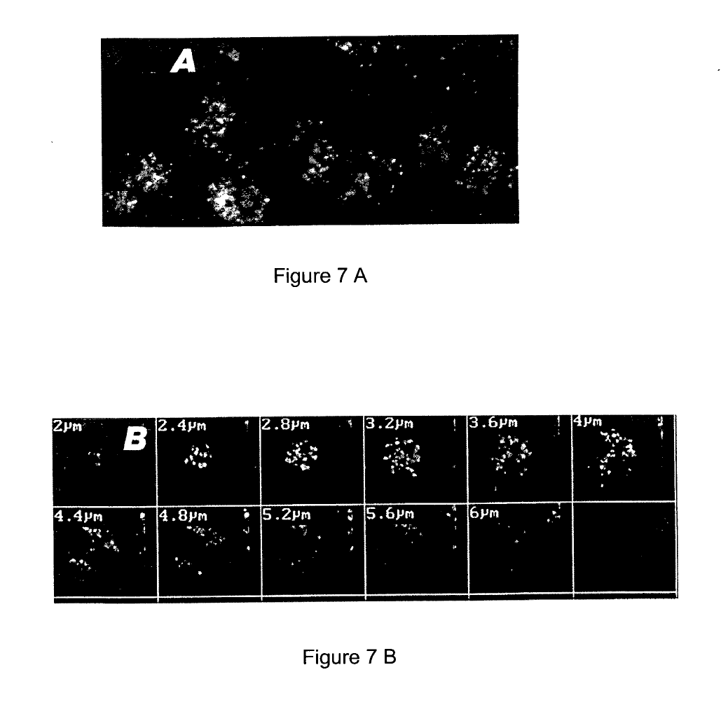

OxyHb-FLEX binding to unstimulated, LPS and TNFα-stimulated (100 & 250 μg/mL) and PMA-stimulated PMNs (30, 100 & 250 μg/mL) increased incrementally with oxyHb-FLEX concentration (FIG. 6). OxyHb-FLEX bound to and internalized into PMA-stimulated PMNs was 2-6 fold times that of unstimulated PMNs at concentrations≧30 μg/mL. Pretreating PMA-activated PMNs with human albumin at levels found in extracellular space of tissues (330 μM; 20 mg/mL) did not alter oxyHb binding (data not shown). Whole PMN and Z-plan fluorescent images (0.2 mM) of PMA-activated PMN treated with oxyHb-FLEX (250 μg/mL) showed clusters of localized oxyHb-FLEX throughout the cytosol (FIG. 7).

### Example 15

### Effects of NO Inhibitors and Antioxidants on DCFH Oxidation

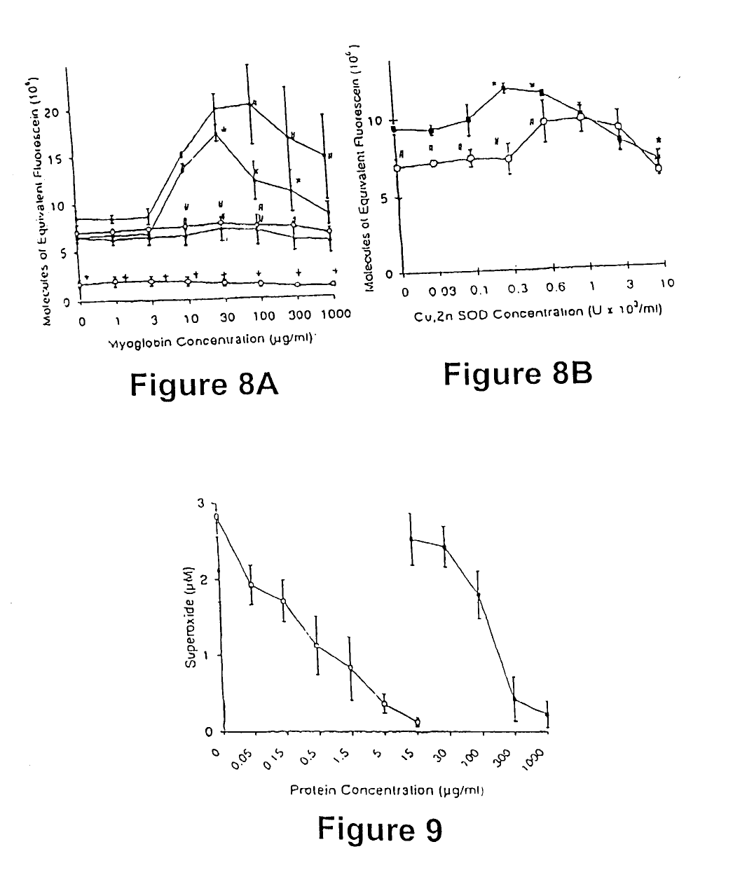

As with unstimulated, and LPS and TNFα-stimulated PMNs, L-NMMA eliminated oxyMb-induced increases in oxidant production in PMA-stimulated PMNs (FIG. 8A). L-NIO, a non-competitive inhibitor of NOS, mirrored the effect of L-NMMA. Physiological levels of glutathione (25 μM) and ascorbate (25 μM) further reduced DCFH oxidation levels below L-NMMA- and L-NIO-inhibited levels, suggesting that a part of the DCFH oxidation is due to H₂O₂ or other oxidants beside NO or that inhibition of NO synthase was incomplete. Human albumin at levels found in the extracellular space of tissues also reduced oxyMb-induced DCH fluorescence by approximately 75%. This level of albumin (20 mg/mL), which is 20-fold greater than the highest heme protein concentration (1 mg/mL), may have partially reduced DCF fluorescence by sequestering PMA from activating PMNs. However, in contrast to glutathione and ascorbate, oxyMb (100 μg/mL) in the presence of albumin still induced DCFH fluorescence 5 times control levels (data not shown).

The superoxide scavenger, human erythrocyte Cu, Zn SOD (300 U/mL, 80 μg/mL), increased oxyMb induced-DCFH oxidation (FIG. 8A). Bovine liver SOD (30-10000 U/mL) produced a biphasic, concentration-dependent increase in DCFH oxidation, with 300 U/mL SOD producing peak levels in the presence of 30 μg/mL of oxyMb (FIG. 8B). This concentration of oxyMb was selected because it consistently generated peak levels DCF fluorescence when used alone (FIGS. 3, 4 and 8A). At SOD concentrations≦600 U/mL, L-NMMA eliminated the SOD-induced increases in DCF fluorescence. The highest concentrations of bovine liver SOD (10,000 U/mL; 26,300 μg/mL) reduced DCF fluorescence below control levels. Bovine liver SOD and oxyMb both exhibited a concentration-dependent reduction of extracellular O₂.⁻ produced by PMA-stimulated PMNs, with SOD being 100-1000 fold more potent (FIG. 9).

### Example 16

### NO Effects on Phosphatidylserine Externalization on Plasma Membrane, DNA Strand Breaks, Plasma Membrane Integrity, and ATP Levels in PMA-stimulated PMNs and Lymphocytes

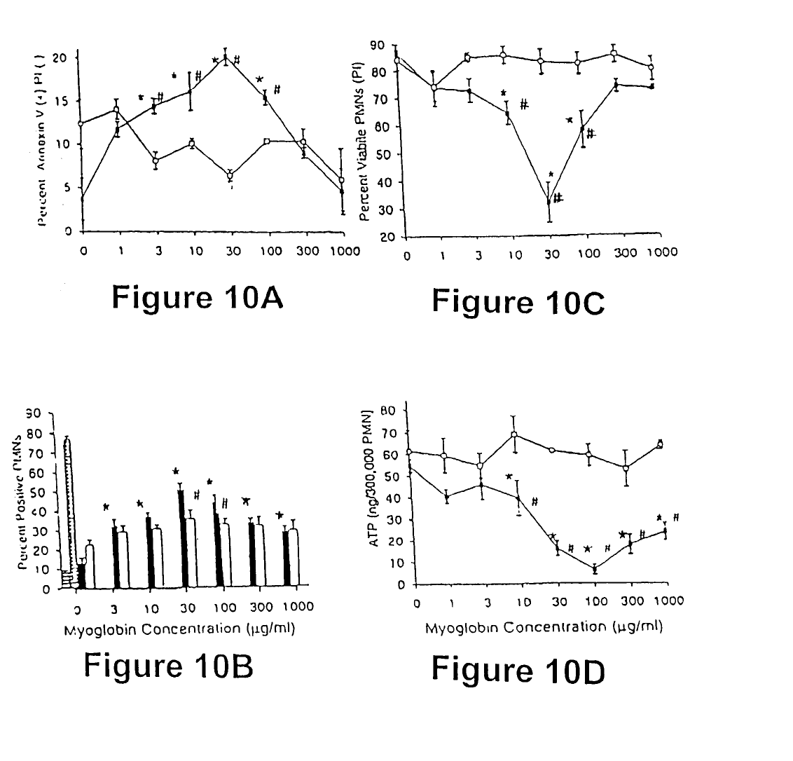

In each of these endpoints, the oxyMb-induced incremental changes in NO. levels paralleled incremental changes in phosphatidylserine externalization on cell membranes (FIG. 10A), DNA strand breaks (FIG. 10B), plasma membrane integrity (FIG. 10C), and intracellular ATP levels (FIG. 10D). Phosphatidylserine positive PMNs were 5-fold (20% vs 3.7%) greater in PMNs treated with oxyMb (30 μg/mL) as compared with PMNs without oxyMb. L-NMMA eliminated 68% of the oxyMb (30 μg/mL)-induced increases in phosphatidylserine levels on PMNs plasma membranes. PMNs containing DNA strand breaks were 4-fold (50% vs 12%) greater in PMNs treated with oxyMb (30 μg/mL) as compared with PMNs without oxyMb. L-NMMA reduced the oxyMb (30 μg/mL)-induced increase in DNA strand breaks by 30%. PMN plasma membrane damage (i.e., reduced viability) was 3-fold (87% vs 32%) higher in PMNs treated with oxyMb (30 μg/mL) as compared with PMNs without oxyMb. L-NMMA eliminated 97% of the oxyMb (30 μg/mL)-induced decreases in PMNs viability. Intracellular ATP levels were 9-fold lower (6 ng vs 55 ng/300,000 PMNs) in PMNs treated with oxyMb (30 μg/mL) as compared with PMNs without oxyMb. L-NIO eliminated 100% of the oxyMb-induced decreases in ATP concentration. Just as 30 μg/mL of oxyMb resulted in the highest concentration of NO., this concentration of oxyMb also produced the most adverse effect on all four apoptotic measures.

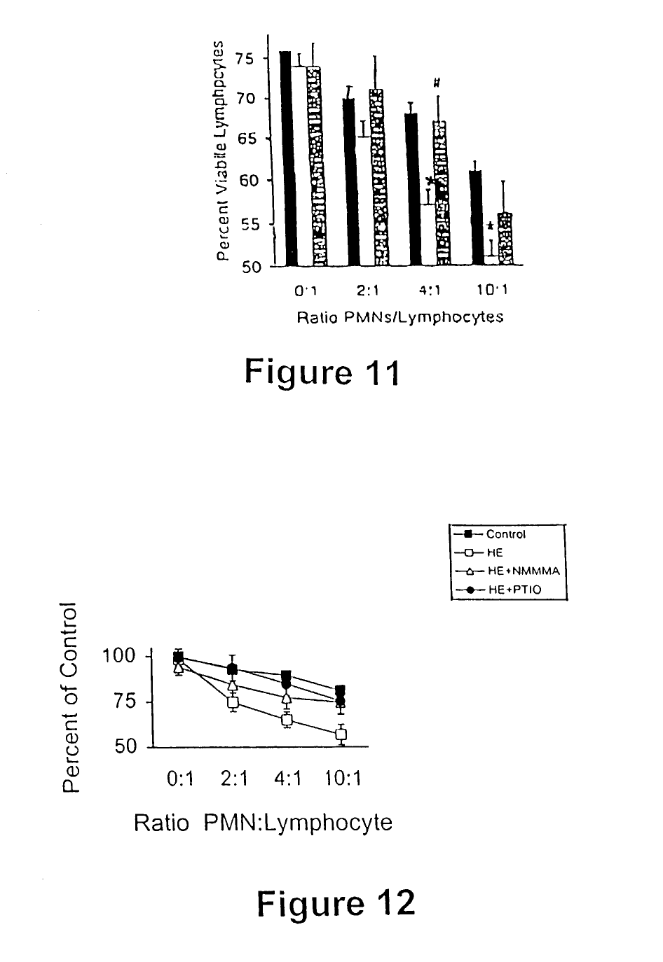

A mixed PMN/lymphocyte preparation was used to determine if the NO. produced by PMNs could affect the plasma membrane integrity of neighboring lymphocytes. PMA-activated and oxyMb (30 μg/mL)-treated PMNs decreased lymphocyte plasma membrane integrity as the ratio of PMNs/lymphocyte increased from 0:1 (76%) to 4:1 (68%) to 10:1 (61%). Preincubation of PMNs with L-NMMA before activation with PMA and oxyMb eliminated the PMN-induced increases in plasma membrane damage in lymphocytes (FIG. 11). These results support the premise that NO originating from PMNs causes cellular damage to neighboring cells.

### Example 17

Human PMNs were selected as a model to assess the effect of oxyHb or oxyMb on cells that produce both NO. and O₂.⁻. To mimic the extravascular environment in which activated PMNs release oxidants and cause cellular damage, PMNs were isolated and washed to remove RBCs, RBC ghosts and their cellular components (e.g., free oxyHb, superoxide dismutase, phospholipids, and catalase). Rejecting PMN preparations containing RBC contamination above 1% and removing visible RBCs and RBC ghosts on top of PMN preparation resulted in a consistently higher yield of NO. from PMA-stimulated and oxyMb-treated PMNs.

### Example 18

Our study shows that oxyMb concentrations produced a concentration-dependent, biphasic effect on DCFH oxidation (i.e., increased NO levels) in unstimulated, LPS and TNFα, and PMA-stimulated PMNs. In each of these conditions, increasing concentrations of oxyMb up to 30 μg/mL incrementally increased DCFH oxidation. Higher concentrations of oxyMb resulted in a reduction in DCFH oxidation with the highest concentration of oxyMb (1000 μg/mL) returning DCFH oxidation to control levels. In PMA-stimulated PMNs, oxyHb produced a similar profile but the DCFH oxidation levels were lower as compared to equal concentrations of oxyMb (i.e., equimolar with respect to heme). S-nitrosylation of the free sulfhydryl group on oxyHb has been reported to reduce NO. levels as compared to oxyMb, which does not contain a free sulfhydryl group. Heme protein effects appear to be dependent on the molar concentration of heme moieties. Since the molecular weights of the subunits of oxyHb and oxyMb are approximately equal, data expressed in concentration (μg/mL) of heme proteins have approximately equal moles of heme. In contrast, concordant amounts of oxyHb encapsulated in a cell membrane (i.e., RBCs) did not increase DCFH oxidation but did reduce DCFH oxidation below that of control values at concentrations above 30 μg/mL. However, sonication of RBCs resulted in increased DCFH oxidation intermediate between intact RBCs and purified oxyHb. The decrease in DCFH oxidation (i.e. NO levels) in PMNs incubated sonicated RBC, as compared to comparable amounts of free oxyHb in the sonicated RBCs, could be attributed to other constituents of RBCs such as SOD and catalase (data not shown). The heme protein-induced increases in DCFH oxidation were not due to ferrous iron since equal molar concentrations of Fe²⁺ resulted in a reduction in DCFH oxidation. In PMN preparations without arginine, equal concentrations of heme proteins produced similar DCFH oxidation profiles regardless of the oxidation state of heme iron or chemical modification (i.e.,α-α diaspirin cross-linked oxyHb). However, metHb was the only heme protein that did not significantly increase DCFH oxidation.

### Example 19

Activation of PMNs increases oxyHb binding to the PMN's cell membrane with subsequent oxyHb internalization. The binding/internalization of heme proteins into PMNs parallels heme protein-induced increases in NO levels and cytotoxic effects in PMNs (Compare FIGS. 4, 5, 6, 10). Kim et al. (Arch. Biochem. Biophy. 309, 308-314 (1994)) have reported that oxyHb binds to PMNs in 30 min (as was true in our experiments) and that oxyHb binding increased several fold in preparations of PMA-activation PMNs compared to unstimulated PMNs. We have also observed that encapsulated oxyHb (i.e., intact RBC) did not increase NO. levels in activated PMNs as compared to sonicated RBCs or a comparable amount of free oxyHb, suggesting that free oxyHb must interact with PMNs to increase NO levels and cytotoxic effects. Kim et al. reported that oxyHb (2 μM; 128 μg/mL) incubated with PMA-activated PMNs reduced extracellular O₂.⁻ levels and PMN viability (trypan blue exclusion) and increased lipid peroxidation of PMNs and survival of extracellular E. coli. The effects of heme proteins in different tissue compartments (e.g., extracellular, membrane-bound, and intracellular) have on PMN oxidant levels and cytotoxicity are unknown and merit further study. Therapeutic agents that inhibit heme protein binding/internalization into PMNs and other cell types that produce oxidants may have clinical value.

### Example 20

The heme protein-induced increases in DCFH oxidation in PMNs appear to be NO mediated. Two NO. synthase inhibitors, L-NMMA, a competitive inhibitor, and L-NIO, a noncompetitive inhibitor, eliminated the oxyMb-induced increase in DCFH oxidation in PMA-stimulated PMNs. Additionally, NO. levels in PMA-stimulated PMNs were markedly increased by the addition of physiological levels of arginine to HBSS. Moreover, the increase in DCFH oxidation in PMNs paralleled total nitrite concentrations in PMN filtrates. Imrich and Kobzik also observed a direct relationship between DCFH oxidation and nitrite levels (Nitric Oxide: Biol. Chem. 1, 359-369 (1997)). Taken together, these effects indicate that NO. is the primary oxidant increasing DCF fluorescence in heme protein-treated PMNs and that a percentage of NO. is oxidizing oxyMb to metMb and forming nitrates.

### Example 21

We have demonstrated that oxyMb-induced increases in NO levels can be eliminated by physiological levels of ascorbate (25 μM) and glutathione (25 μM), and reduced by extracellular levels of albumin (330 μM; 20 mg/mL). Reduction in NO. levels by glutathione or albumin could be the result of s-nitrosylation of an available cysteine residue in each thiol protein. Due to the probability of a larger interstitial space (i.e., extracellular volume) in our preparations as compared to tissues, the antioxidant capacity will likely be greater than in vivo. Even during inflammation when the extracellular volume in tissues may double, the extracellular volume is less than the volume of the PMNs. In our preparations, PMNs are allowed to settled to the bottom of a round bottom tube containing buffer that is 500 times the volume of the PMNs while being rocked in a water bath.

### Example 22

We propose the following model to account for the bell-shaped, concentration-dependent heme protein effects on intracellular levels of NO. Upon activation, NADPH oxidase of PMNs releases the negatively charged O₂.⁻ to the cell exterior where it interacts with heme proteins. Internalized heme proteins could also interact with O₂.⁻ produce by PMN vacuoles. Since the reaction of O₂.⁻ with NO. to form ONOO⁻⁰ is extremely rapid (3.7×107 M⁻¹ S⁻¹), it is conceivable that heme proteins (3-30 μg/mL) bound to plasma or vacuole membranes bind and/or dismute O₂.⁻, thereby increasing the NO concentration by limiting the scavenging action of O₂.⁻. This model is consistent with other reported data showing that PMNs activated with the PMA concentration used in this investigation produced twice the number of moles of O₂.⁻ as NO. and that low concentrations of SOD increase NO. levels. Therefore, at low concentrations of heme proteins, NO levels are increased by virtue of the heme binding and/or the dismuting of O₂.⁻. However, at concentrations of oxyMb above 30 μg/mL, additional heme proteins located at the cell surface, internalized or in solution removed NO. to a greater extent, thereby abolishing the effect of lowered O₂.⁻. In experiments conducted at atmospheric oxygen levels, heme proteins are almost fully oxygenated. OxyHb (24×10⁶ M⁻¹S⁻¹) or oxyMb (17×10⁶ M⁻¹S⁻¹) rapidly reacts with NO to produce metHb or metMb and nitrate. Internalized heme proteins could form the anion, nitrate, in PMNs, thereby reducing extracellular levels of nitrates. We observed that nitrite/nitrate levels in filtrates of PMA-activated PMNs progressively decreased at oxyMb concentrations above 30 μg/mL.

### Example 23

Heme proteins mimicked Cu, Zn-SOD in dismuting extracelluar O₂.⁻ and in increasing intracellular NO. levels. The concentration-dependent profiles for eliminating extracellular O₂.⁻ were similar for oxyMb and SOD but the oxyMb profile was shifted 2 orders of magnitude to the right. Also, human erythrocyte (300 U/mL; 800 ug/mL) and bovine liver Cu, Zn-SOD (300-600 U/mL) increased intracellular NO levels in PMA-stimulated PMNs beyond oxyMb-treatment only. Like heme proteins, SOD induced a biphasic, concentration-dependent alteration in NO. levels, with the highest concentration of SOD reducing fluorescence below control levels. At the lower SOD levels, L-NMMA eliminated the SOD-induced DCF fluorescence, suggesting that SOD increased NO levels and not H₂O₂ levels. These SOD-induced NO levels may be attenuated at higher SOD levels by the formation of s-nitrosothiol in a similar manner as in oxyHb.

### Example 24

Several reports have shown that the human Cu, Zn-SOD expressed in mice or cell lines potentiates oxidant damage that leads to cellular damage and death. Transgenic mice over-expressing human extracellular superoxide dismutase by 5-fold in brain tissues showed a dramatically increased mortality (83%) compared to non-transgenic littermates (33%) upon exposure to 6 atm of hyperbaric O₂ for 25 min. The increased mortality was eliminated by either diethyldithiocarbamate, which inhibits SOD, or N-nitro-L-arginine, a NOS inhibitor. These transgenic mice also had higher lipid peroxidation and abnormal neuromuscular junctions. Furthermore, the biphasic Cu, Zn-SOD effect was reported in a human hepatoma liver cell line (HepG2) treated with 3-morpholinosynonimine (SIN-1), which releases both NO. and O₂.⁻. Cu, Zn-SOD potentiated SIN-1 cytotoxicity in a concentration dependent manner up to 100 units of Cu, Zn-SOD/mL but decreased toxicity at higher concentrations. These reports support our contention that compounds dismuting and/or sequestering O₂.⁻ can increase cellular damage by preventing O₂.⁻-mediated inactivation of No.

In our study, oxyMb-induced increases in NO. levels paralleled increases in several cellular alterations associated with apoptosis in PMNs: phosphatidylserine externalization to the outer surface of the plasma membrane, cell membrane damage, DNA strand breaks and decreases in cellular ATP levels. In each of the four apoptotic measures, oxyMb-induced NO. effects were reversed by NOS inhibitors. Although the effects of oxyHb on these apoptotic measures were not assessed we would predict similar results.

### Example 25

Externalization of phosphotidylserine from the inner leaflet of the plasma membrane to the outer surface has been reported to be an early marker for apoptosis in human PMNs initiated by several apoptotic agents. OxyMb-induced externalization of phosphatidylserine was observed after 1 h of treatment, prior to plasma membrane damage detected by propidium iodide (PI). NO. effects on membrane integrity could be attributed to direct oxidation of membrane components and/or decreased cellular levels of ATP required for maintaining the cell membrane function and integrity. NO. has been reported to inhibit cellular enzymes and macromolecules containing Fe—S clusters or heme groups, such as mitochondrial aconitase activity, as well as complexes I and II, and cytochrome oxidase of the electron transport chain. NO.-inhibition of any of these Fe-proteins could reduce ATP synthesis.

### Example 26

NO has also been shown to cause DNA single strand breaks [61] and DNA deamination and to inhibit DNA synthesis via inhibition of ribonucleotide reductase. These NO-induced DNA strand breaks activate poly (adenosine 5′-diphosphoribose) synthetase that further depletes cellular ATP levels. Increasing the level of O₂.⁻, by adding SIN-1 or 12-O-tetradecanoylphorbol-13-acetate (TPA), markedly reduced the level of DNA fragmentation. Similar results were observed in rat PMNs and in a cell free system where increasing O₂.⁻ levels reduced the N-nitrosation of primary aromatic amines which was reversed by addition of SOD. Taken together, these results indicate that NO.-induced apoptosis in intact cells is reduced when NO. is scavenged with increasing concentrations of O₂.⁻ and would demonstrate a NO. regulating role for O₂.⁻.

### Example 27

The concentration-dependent effect of PMA-activated and oxyMb-treated PMNs on the integrity of the plasma membrane of co-incubated lymphocytes reinforces the previous observation that toxic levels of NO. are originating from neighboring PMNs. The ability of L-NMMA to reverse the effects of activated-PMNs on lymphocyte membrane integrity further supports this contention.

### Example 28

Elevated levels of free heme proteins have been reported in transfused blood and in the vasculature during numerous pathologies. Hemolysis of RBCs in stored blood increases with time and results in approximately 1% hemolysis after 35 days. OxyMb and oxyHb levels in serum of severe burn patients frequently reach levels of 3-30 μg/mL [1-3]. Similar levels have been reported in patients with rhabdomyolysis (10 μg/mL) and crush injuries (30 μg/mL). Lower levels (1-3 μg/mL) have been reported in serum of patients after myocardial infarction. However, higher myoglobin levels would be predicted in areas surrounding damaged myocardial and skeletal muscle tissue. In this study, levels of heme proteins found in the vasculature during various pathologies increased intracellular levels of NO. and cytotoxicity in activated PMNs and neighboring lymphocytes.

### Example 29

The role of hemolysis of RBCs in transfused blood in the predisposition to infections, multiple organ failure, and recurrence of cancer has been established but is not understood. Lysed RBCs not only release Hb but also Cu, Zn SOD, catalase, phosphatidyserine and phosphatidylethanolamine from the inner leaflet of the plasma membrane. Phosphatidyserine and phosphatidylethanolamine treated macrophages increased oxidant levels and lipid peroxides in the incubation media. Plasma Hb in transfused whole blood has been measured as high as 440 μg/mL and as high as 5,780 μg/mL in red cell concentrates. At these levels of free Hb, it is evident that transfusions could decrease extracellular O₂.⁻ levels around cells that produce NO. and O₂.⁻ thereby, increasing the potential for NO-mediated cytoxic effects on neighboring cells. Our proposed model of heme protein-induced damage to host defense and other cells may explain transfusion-induced increases in infection, tissue damage and recurrence of cancer.

### Example 30

Survival after severe burns as after other types of trauma is age-dependent and biphasic, peaking in young adults and decreasing markedly in prepubescent and older adult patients. The biphasic, age-dependent survival in trauma patients mirrors plasma levels of 17β-E2, testosterone (T) and dehydroepiandrosterone (DHEA) in women, and of T and DHEA in men. DHEA and T are readily converted to 17β-E2 by aromatase, whose expression and activity in tissues is increased by inflammatory cytokines following trauma. Chlordane, the parent compound of HE and OC, has been shown to inhibit aromatase.

### Example 31

Recently, 17-β E₂ has been shown to attenuate reperfusion injury in a variety of models by reducing the inflammatory response. 17-β E₂ reduces the levels of circulating adhesion molecules, leukocyte adherence, and the transendothelial migration of leukocytes. Similar beneficial effects of 17-β E₂ have been reported in post menopausal women. 17-β E₂ has also been reported to reduce the increased release of inflammatory cytokines (TNFα, II-1 α & β, and GM-CSF) by monocytes or macrophages following LPS-stimulation, reperfusion injuries in rats, oophorectomy, or menopause. Physiologial levels of 17-β E₂ also inhibits cytokine-induced expression of inducible nitric oxide (NO) synthase and the resulting high nitric oxide production in macrophages, endothelial, and smooth muscle cells. The attenuation of NO levels by 17-β E₂ may be partially due to the inhibition of hexose monophate shunt enzymes that metabolize glucose to NADPH required for the syntheses of oxidants.

### Example 32

HE effects on intracellular levels of oxidants in PMNs were measured using a previously reported cytofluorometric procedure. Briefly, PMNs were loaded with 2′,7′-dichlorodihydrofluorescein diacetate (DCFH-DA)(Kodak, Rochester, N.Y.) at 37° C. for 15 min followed by a 10 min incubation with and without nitric oxide (NO) synthesis inhibitor, N-methyl-L-arginine (L-NMMA) (5 mM) (Calbiochem, San Diego, Calif.), 1β(-E2 receptor antagonists, tamoxifen (200 nM) (Sigma, St. Louis, Mo.) and ICI 182,780 (0.1 nM) (Zeneca Pharmaceuticals, Cheshire, England), and TNFα (10 ng/mL) (Sigma, St. Louis, Mo.). All PMNs were subsequently incubated with varying concentrations of HE for 40 min (Ultra Scientific, Kingstown, R.I.). Cytofluorometric analyses of oxidized DCFH in PMNs were performed with a FACScan (Becton Dickinson, San Jose, Calif.).

HE-induced DNA strand breaks in PMNs were measured by flow cytometry using the tdt-mediated dUTP nick end labeling assay, In Situ Cell Death Detection Kit (Boehringer Mannheim, Indianapolis, Ind.) according to manufacturer's instructions. PMNs were incubated with L-NMMA, 17β-E2 receptor antagonists, TNFα, and varying concentrations of HE as described in the above HE-induced DCFH oxidation experiments with the exception that HE incubation time was 2 h instead of 40 min.

HE effects on surrounding lymphocyte membrane integrity mediated by PMN-derived NO were determined by cytofluorometric measurements of intracellular propidium iodide levels in a mixed PMN/lymphocyte preparation using 100,000 lymphocytes and PMN/lymphocyte ratios of 0/1, 2/1, 4/1, 10/1). For comparison some aliquots of PMNs were loaded with L-NMMA (5 mM) or 2-(4-carboxyphenyl)-4,4,5,5-tetramethyl imidazoline-1-oxyl-3-oxide (carboxy-PTIO) (5 mM) (Calbiochem, La Jolla, Calif.), a NO scavenger, prior to incubating leukocytes with HE (1 nM) for 40 min.

### Example 33

### Organochlorines in Serum

Serum levels of HE and OC in nonsurviving burn patients were highest on PBD 5 and returned almost to PBD 1 levels by PBD 11. In contrast, serum levels of HE and OC were not altered in surviving patients. Mean peak serum levels of HE (215 pg/ml) (0.55 nM) (P<0.05) and OC (677 pg/ml) (1.59 nM) in nonsurviving patients on PBD5 were approximately 90% higher than in surviving patients and greater than 200% those of normal subject values. When data were expressed as the mean for samples from PBD 3-7, representing the post-injury hypermetabolic phase, HE levels in non-survivors were higher than survivors (P<0.05) and normal subjects HE (P<0.002) and OC levels were higher than in normal subjects (P<0.005). In comparison, mean DDE levels in nonsurvivors were greater and more variable, increasing 2.5 times to 3667 pg/mL (183 nM) by PBD 11, but were not significantly different from surviving patients nor normal subjects. The increased variability of DDE may be due to increased affinity for serum lipids. DDE has a n-octanol/water partitioning coefficient (log K_{ow}) of 5.69 that is over an order of magnitude greater than the log K_{ow} for cyclodienes (4.32). The continued increase in DDE levels for PBD 7 & 11 may be due to the reduced levels of serum lipids, allowing increased extraction efficiency. Mean HE, OC, and DDE levels in surviving patients were within one standard deviation of the mean of normal subjects. We measured serum levels of cholesterol and triglycerides to assess whether increased levels of lipophilic xenoestrogens could be attributed to increased serum lipid levels. There was no difference in cholesterol and triglycerides between the surviving and nonsurviving burn patients. However, patients with higher serum lipid levels had higher levels of HE and OC (P<0.05). Serum levels of cholesterol decreased markedly following injury (P<0.001), whereas mean triglycerides exhibited nonsignificant variation. Decreasing serum lipids may increase the availability of these xenoestrogens to disrupt receptor-mediated processes. We did find strong intercorrelations among levels of HE, OC, and DDE in serum (r=0.59-0.87, all P<0.001). How much of this correlation is due to serum lipid levels or to the interrelationships in exposure to these two classes of organochlorine insecticide is unknown. Average body weights of surviving and nonsurviving burn patients were almost identical for the first 4 days following injury, indicating that both groups of patients had similar resuscitation and that resuscitation was not responsible for alterations in serum levels of organochlorines.

Regression analysis accounting for age, burn size, serum lipids and survival showed a coincident increase in HE, OC, and DDE levels in serum with age (P<0.01). An unexpected observation was that serum levels of HE (P<0.01) and OC (P<0.05) were better predictors of mortality in burn patients than age or burn size. These results are at least compatible with a casual relationship between serum insecticides and survival in burn patients. We therefore conducted in vitro studies to ascertain whether HE, at levels found in the serum of burn patients, would increase oxidants and oxidative damage in isolated human PMNs and NO-induced damage to neighboring cells.

### Example 34

### Endocrine Disruptor Effects on Oxidant Production on PMNs

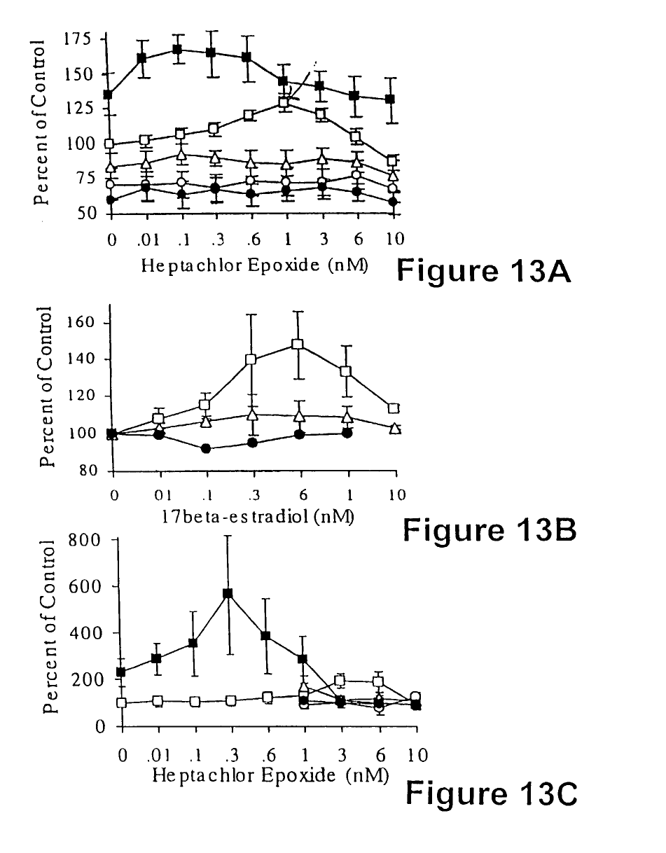

HE increased the intracellular level of oxidants (P<0.001) in PMNs in a concentration-depend, biphasic manner (FIG. 13a). This inverted U response of intracellular oxidants was fitted to a Gaussian curve (P<0.001) with a peak (maximal) response of approximately 1 nM (10-8.93 M). Adding L-NMMA to HE-treated PMNs completely inhibited the oxidation of intracellular DCFH (P<0.001), indicating that NO was the predominate oxidant species. Augmenting PMN-treated HE with TNFa caused a 60% increase in amplitude (P<0.001) and a 10-fold left shift of the Gaussian curve (P<0.01), peaking at approximately 0.1 nM (10-10.20 M). Co-incubating PMNs with HE and either ICI 182,780 or tamoxifen completely eliminated the HE-induced increase in NO (P<0.001). 17β-E2 at similar concentrations produced similar inverted U Gaussian response curves for intracellular oxidants, peaking at approximately 0.6 nM (10-9.32 M) (P<0.001) (FIG. 13b) compared with approximately 1 nM for HE (FIG. 13a). Adding L-NMMA to 17β-E2-treated PMNs attenuated intracellular oxidants (P<0.01) also indicating that NO was the major oxidant. As in HE-treated PMNs, tamoxifen completely inhibited the 17β-E2-induced NO levels (FIG. 13b)(P<0.001).

### Example 35

### Endocrine Disruptor Effects on Cellular Damage in PMNs

HE increased the level of DNA strand breaks in PMNs in a concentration-depend, inverted U manner. This inverted U response of intracellular DNA damage was fitted to a Gaussian curve (P<0.001) exhibiting a peak response for HE at approximately 5 nM (10-8.51 M) (FIG. 13c). Augmenting HE with TNFα caused a 445% increase in amplitude (P<0.05) and a 10-fold left shift of the Gaussian curve (P<0.01), peaking at approximately 0.3 nM (10-9.58 M). Co-incubating PMNs with HE and either L-NMMA, ICI 182,780, or tamoxifen showed a nonsignificant trend for attenuating HE-induced DNA strand breaks at the HE-induced peak values (3-6 nM).

Incubating a mixed PMN/lymphocyte preparation with HE (1 nM) increased cell membrane damage in the lymphocytes at PMN/lymphocyte ratios above 2 (P<0.001) (FIG. 12). Co-incubation of PMN/lymphocyte preparations with HE and L-NMMA or PTIO, a NO scavenger, attenuated the HE-induced cell membrane damage in the lymphocytes (P<0.05). These results indicate that HE-induced levels of NO can cause cellular damage to neighboring cells.

### Example 36

In non surviving burn patients, the serum levels of the cyclodienes, HE and OC, were elevated with peak values of PBD 5 during the first week following injury as compared to age-and burn size-matched surviving patient and normal subject values. This time course mimics other markers of inflammation, and tissue injury at their peak values of PBD 5. A trend for increased serum levels of DDE above those in surviving and normal subjects was also observed, however, serum levels of DDE did not decline after PBD 5. In a dietary restriction study that reduced the body fat in rats by 50%, dieldrin, another similar cyclodiene insecticide, was mobilized from the fat stores whereas DDE levels doubled in the remaining fat. These results suggest that different classes of organochlorine insecticides can be differentially mobilized during lipolysis. In burn patients, serum levels cholesterol decreases with PBD and triglycerides increase with the severity of the burn peaking between PBD 4-6.

### Example 37

During the first week following severe burn injury, serum levels of TNFα increase, reaching peak levels on PBD3-5. TNFα levels increase with the severity of the burns and are higher in non surviving patients. TNF treatment increases lipolysis and fat utilization in cancer patients and in in vitro models. TNF-induced lipolysis would release sequestered estrogenic compounds, e.g., HE and OC, from adipose tissues. These bioavailable estrogenic compounds could increase the metabolism of 17-βE₂ and block the 17-βE₂ receptors attenuating 17-βE₂ inhibitory effect of TNF levels resulting in a positive feedback loop with increasing levels of TNF, lipolysis, and HE and OC. These insecticides may be acting as more than markers of the hypermetabolic state, causing either increased lipolysis only or increased lipolysis with adverse effects in burn patients due to increased bioavailable HE and OC levels.

### Example 38

### Criteria for Subject Selection

Patients and Subjects. Analyses of xenoestrogens in archived serum were performed on samples from seven nonsurviving males and seven age- and burn-size matched surviving male burn patients admitted between 1991 and 1994 to the United States Army Institute of Surgical Research burn care facility. The criteria for selection of subjects were age (approximately 40-70 years), total burn surface area (≧25%), postburn survival (≧7 days) and lack of preburn disease. Inhalation injury was present in 6 of 7 surviving and 5 of 7 nonsurviving patients. In the non-surviving group, two patients died between postburn day (PBD) 7 and 11. Age-matched healthy males (n=12) were used as control subjects.

### Example 39

### Organochlorines in Serum

Levels of insecticides in serum of burn patients on postburn day (PBD) 1, 3, 5, 7, and 11 were determined in accordance with a previously reported method25. This method used a Dynamic Thermal Stripper (Environchem, Kemblesville, Pa.) to sparge insecticides from 50 μl of serum. The insecticides were collected on Tenax solid sorbent and subsequently thermally desorbed with a Unacon 810 (Environchem, Kemblesville, Pa.) into a HP 5890 gas chromatograph (Hewlett Packard, Palo Alto, Calif.) equipped with a SPB-608 30 M, fused silica capillary column (J & W Scientific, Folsom, Calif.) and an electron capture detector. Levels of insecticides in serum stored at −75° C. were determined by comparing responses to standard curves constructed from certified standards (Ultra Scientific, Kingstown, R.I.) spiked into a composite sample consisting of serum from 3 subjects, previously determined to have low levels of each insecticide. For consistency, each patient's samples were analyzed during the same day by postburn day (PBD 1, 3, 7, and 11). The detection limit for HE and OC was 76 and 56 pg/mL, respectively. A reference serum sample pooled from 3 patients having medium levels of each insecticide was measured 1-2 times during each day of analyses. There was no difference in mean levels of HE, OC, or DDE in reference samples analyzed concurrently with samples from surviving as compared to nonsurviving patients The analytical procedure was optimized for the semivolatile HE and OC with the overall coefficient of variation for reference samples of <20% for HE and OC and 75% for DDE. Serum cholesterol and triglyceride concentrations were determined using Instrumentation Laboratory reagents and analyzed on a Monarch Clinical Chemistry System (Instrumentation Laboratories, Inc., Lexington, Mass.).

### Example 40

### Endocrine Disruptors on Oxidant Production and Cellular Damage

In Vitro measurements of oxidant production and cellular damage. Polymorphonuclear leukocytes (PMNs) and lymphocytes were obtained from EDTA-preserved venous blood of non-smoking adult males by layering blood over Polymorphprep (Nycomed Pharma, Oslo, Norway) as previously described. The purity of PMNs and lymphocytes was greater than 88% and 95%, respectively, and their viability, as determined by trypan blue-exclusion, was greater than 90%. All experiments were conducted with 300,000 PMNs added to 100 μL of Hanks Balanced Salt Solution. All PMNs were incubated with Ca2+ (50 (M) and physiological levels of arginine (100 μM). Replicate experiments were conducted using PMNs isolated from different donors.

### Example 41

In an isolated human PMN model, we found that HE increased NO levels and DNA single strand breaks in inverted U-shaped response curves that peaked close to the levels of HE found in the serum of our burn patients. 17β-E2 produced almost identical response curves with peak levels of NO generated at 0.6 nM and 1.0 nM for 17β-E2 and HE, respectively. HE and 17β-E2 effects on NO levels were inhibited by 17β-E2 receptor antagonists suggesting that PMN-derived-NO was 17β-E2 receptor mediated. Mixed PMN/lymphocyte studies indicated that NO produced by PMNs treated with HE could damage neighboring cells. When HE-treated PMNs were incubated with relevant levels of TNFα, not only did TNFα increase the overall intracellular levels of NO and levels of DNA strand breaks but also shifted the inverted U response curves an order of magnitude to the left for NO levels and for DNA strand breaks. Considering that NO is known to increase levels of inflammatory cytokines60, such as TNFα, a positive feed back loop may develop following trauma, where rising levels of TNFα increase levels of phagocyte-derived NO by left-shifting the NO response curve to lower HE concentrations, exacerbating cellular damage. Since HE and 17β-E2 effects are concentration-dependent, the overall effects of xenoestrogens on 17β-E2 mediated processes may vary depending on the concentration of each xenoestrogen and their interactions.

### Example 42

Depending on their concentration, xenoestrogens may act as agonists to 17β-E2 receptors, inducing steroid hydroxylases that convert 17β-E2 and other steroids to inactive metabolites and inducing oxidants that damage surrounding cells. Simultaneously, xenoestrogens may act as antagonists blocking the 17β-E2 receptor such as induction of aromatase activity thereby, inhibiting 17β-E2 beneficial effects seen in multiple injury models. These xenoestrogens may induce positive feedback loops for lipolysis, and inflammatory cytokine expression, and oxidant production. Each pathway producing mediators that augment the other. Since these xenoestrogens are accumulating in lipids with age, as 17β-E2 levels and its precursors (DHEA and testosterone) are decreasing, the potential for altering 17β-E2 receptor-mediated effects increases with patient age. The increasing xenoestrogens:17β-E2 age-related ratio could contribute in the decrease in survival in older as compared to younger patients at ours and another burn center during the time when these xenoestrogens were used extensively ('60s and '70s), and for the increase in coronary heart disease in the '60s. More recent data reported here for burn patients admitted between 1991 and 1994 suggest that these xenoestrogens are linked to lower survival. A concept that deserves further assessment is whether some of the adverse health effects associated with serum cholesterol and triglycerides may actually be due to elevated levels of lipophilic chemicals bound/transported by these serum lipids. This study lends support to the notion that even very low concentrations of xenoestrogens may have untoward effects, especially following severe injury when xenoestrogens mobilized from fats stores and inflammatory cytokines may act synergistically.

### Example 43

All reagents, unless otherwise stated, were obtained from Sigma Chemical Co. (St. Louis, Mo.). Human PMNs and MNCs were obtained from sodium citrate (0.105 M)-preserved venous blood from nonsmoking, drug free, healthy male volunteers. PMNs and MNCs were harvested from blood via centrifugation with equal volumes of Polymorphprep, GIBCO (Grand Island, N.Y.) at 550×g for 35 minutes at 20° C. PMN and MNC layers were isolated and washed twice in Hank's Balanced Salt Solution (HBSS) (GIBCO, Grand Island, N.Y.). Two hypotonic lyses were performed to lower the RBC contamination of the PMNs and MNCs. The average purity of PMNs and MNCs was greater than 95% and 90%, respectively as determined by use of ABX Pentra 120 cell counter. Replicate experiments were conducted with three to five donors from a pool of six consented male adults.

Intracellular Oxidants and Membrane Damage

PMNs (10,000,000/ml) and MNCs (20,000,000/ml) were loaded with 2,7-dichlorodihydrofluorescein-diacetate (DCFH-DA) (2 μM) (Molecular Probes; Eugene Oreg.) in HBSS for 30 minutes at 37° C. in a water bath. Non fluorescent DCFH is oxidized to fluorescent DCF by a variety of oxidants. The approach is sensitive, reproducible, and capable of measuring wide concentration range of intracellular oxidants. DCFH-loaded PMNs (500,000) or MNCs (1,000,000) in 50 μl HBSS were combined with tubes (12×75 mm) containing 50 μL of either no stimulant, a cytokine cocktail of human TNF-α (2,500 U/mL), IL1-β (500 U/mL) and IFN-γ (1,250 U/mL) (R&D Systems, Minneapolis, Minn.), human hemoglobin (100 μg/mL), combination of cytokine cocktail and hemoglobin stimulation, or phorbol myristate acetate (PMA) (50 nM) (Calbiochem, San Diego, Calif.) used as a positive control. PMNs or MNCs, stimulated with a combination of cytokine cocktail and hemoglobin, were also treated with and without ethanol (0.05%) used to solubilize the highest steroid concentrations; nitric oxide synthase (NOS) inhibitor; N-methyl-L-arginine (L-NMMA) (10 mM); its inactive stereoisomer, N-methyl-D-arginine (D-NMMA) (10 mM); or a selective inhibitor of iNOS, L-N⁶-(-1-Iminoethyl)lysine (L-NIL) (10 μM) (all, Calbiochem, San Diego, Calif.); or 10 equimolar concentrations of either E₂, P₄, cortisol (10⁻⁹ to 10⁻⁵ M); or a combination of 5 E₂ concentrations (6×10⁻⁷ to 10⁻⁵ M) with a single concentration of P₄ (6×10⁻⁷ M). Treated PMNs or MNCs were incubated for 1 or 5 h in HBSS amended with physiological concentrations of Ca²⁺ (1.5 mM), Mg²⁺ (900 μM), glutamine (1.0 mM); GIBCO; Grand Island, N.Y.), arginine (100 μM), amino acids (1×MEM)(GIBCO; Grand Island, N.Y.), and tetrahydrobiopterin (1.0 μM). Flow cytometric analyses were performed with an argon laser (488 nm) and emission light measured behind a filter transmitting 530/30 nm light on a FACCalibur, Becton Dickinson (San Jose, Calif.) with CELLQuest data acquisition and analysis software. PMNs or MNCs were gated by forward- and side-scatter. For each sample, 10,000 PMNs or MNCs were collected. The mean channel fluorescence was determined on a linear scale from a single parameter histogram.

Cell membrane damage in activated PMNs and MNCs was measured by flow cytometry analyses after a 5 min treatment at room temperature with propidium iodide (1.0 μg/ mL). Procedures, treatments and measurement of PI uptake in PMNs and MNCs were identical to those described for intracellular oxidants at 5 h.

Statistical Analyses Intracellular oxidants in activated MNCs or PMNs treated with the three highest steroid concentrations or the L-NMMA treatment were compared to untreated activated MNCs or PMNs by Bonferroni corrected t-test.

### RESULTS

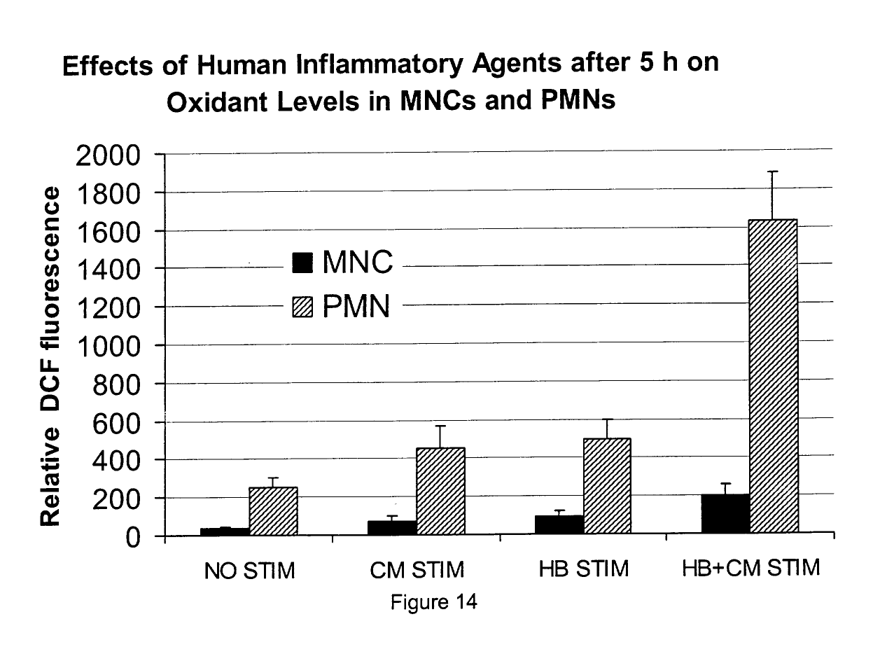

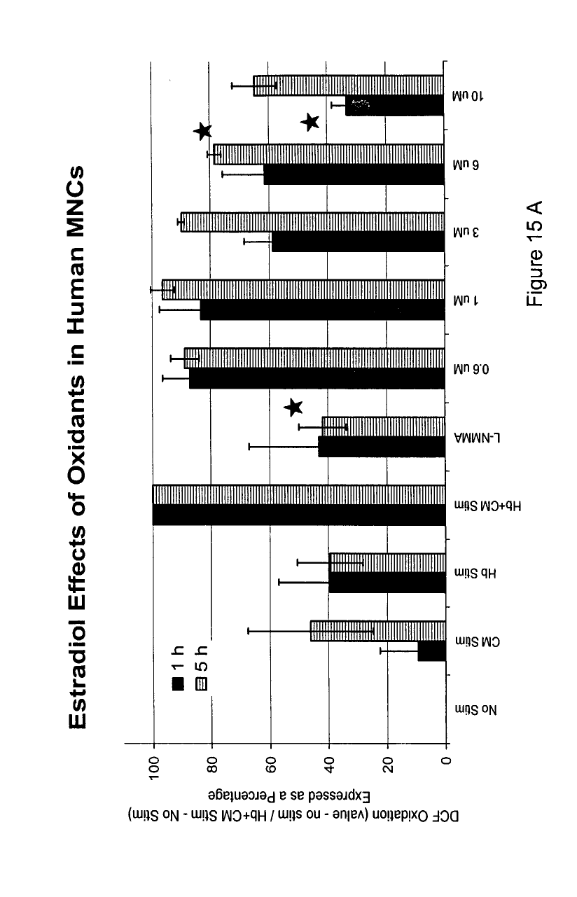

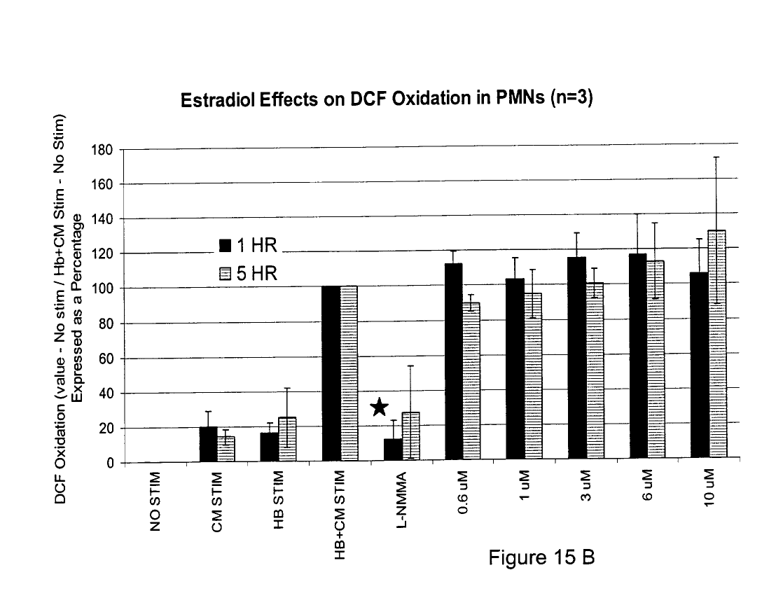

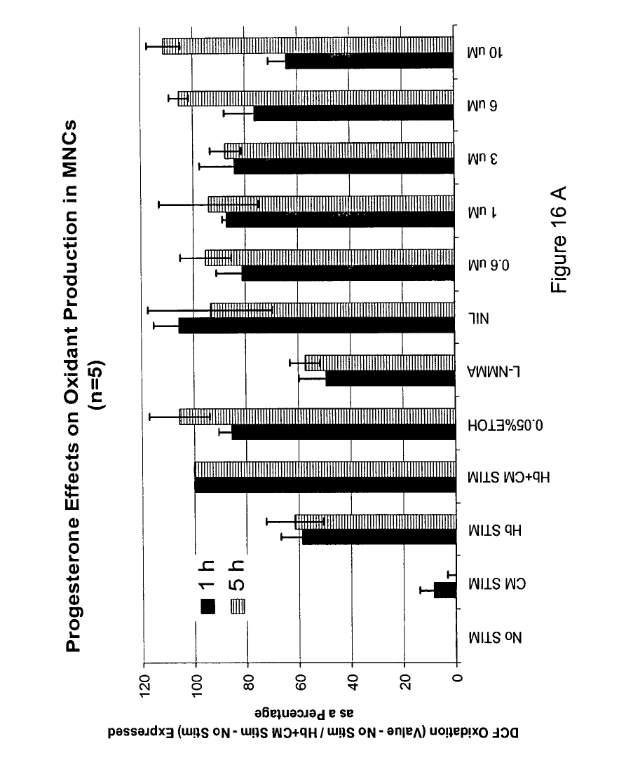

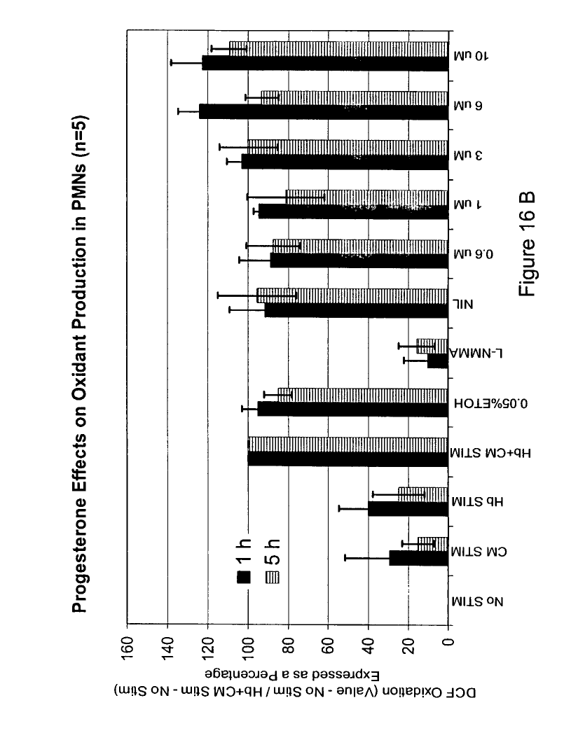

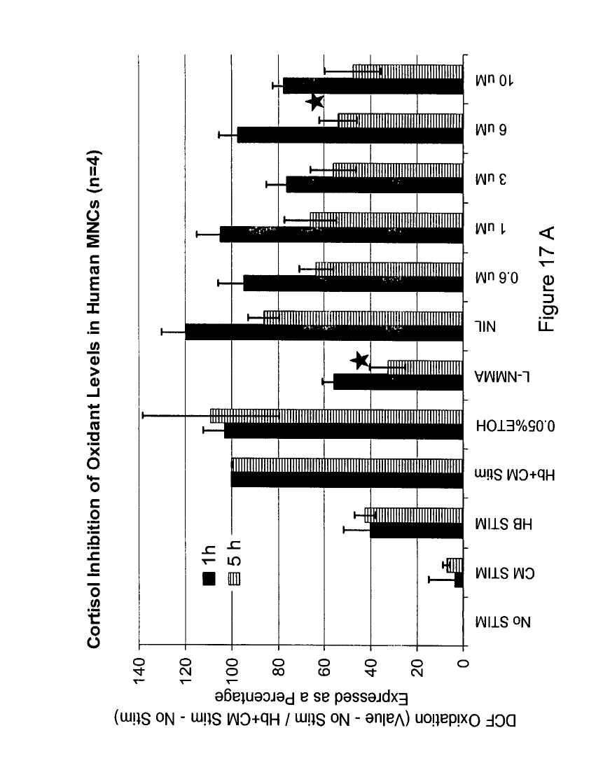

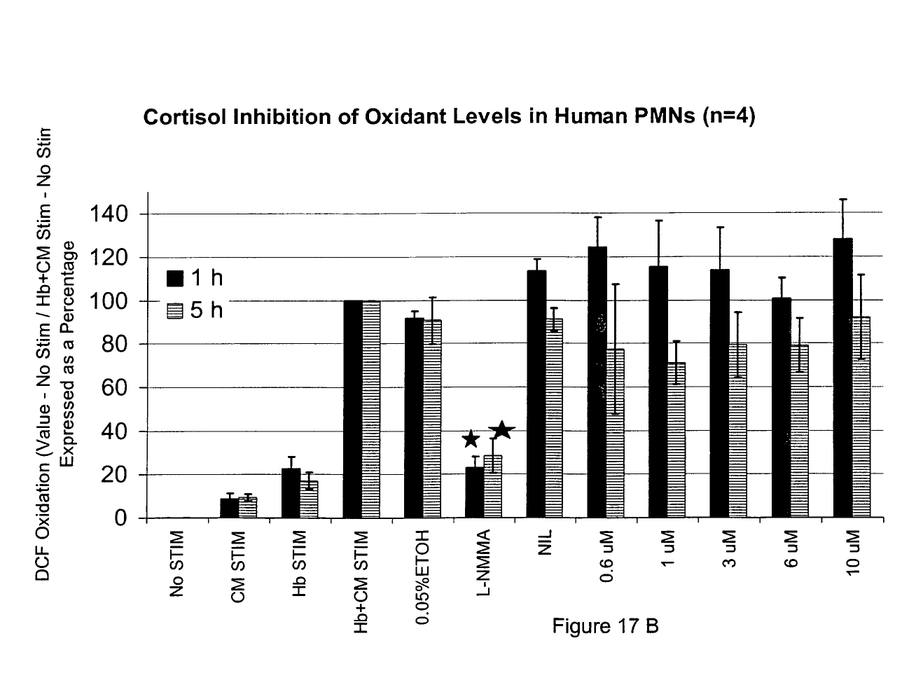

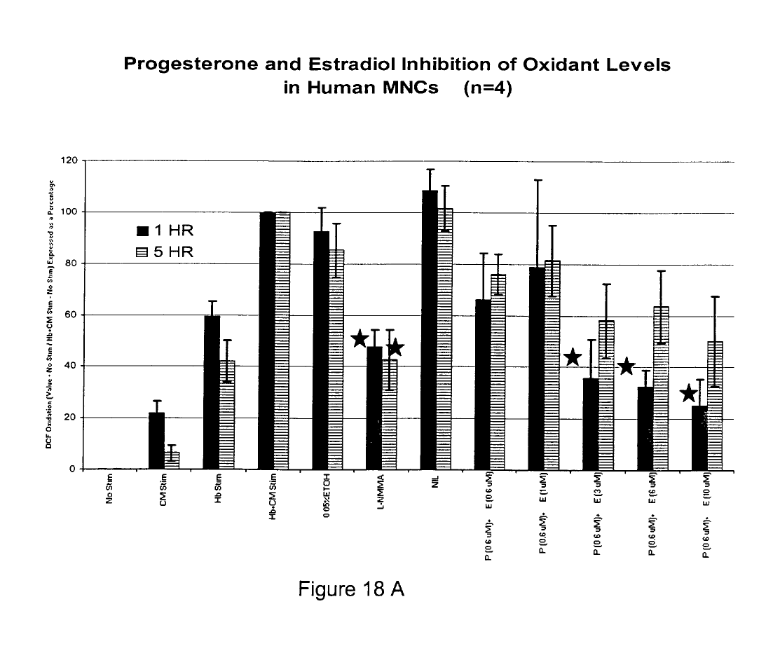

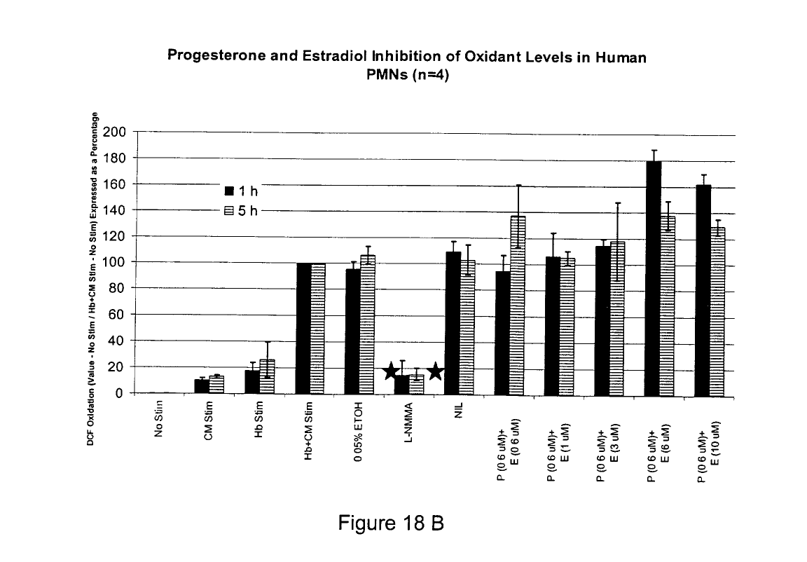

Compared to mean background fluorescence in unstimulated MNCs and PMNs, the mean intracellular levels of oxidized DCF increased 189% and 181% with cytokine stimulation, 259% and 197% with hemoglobin stimulation, and 546% and 648% with a combination of hemoglobin and cytokine stimulation after 5 h in MNCs and PMNs, respectively (FIG. 14). After 1 h of activation, the relative increases in mean oxidized DCF were lower, especially for MNCs with increases of 9% and 91% for cytokine stimulation, 41% and 172% for hemoglobin stimulation, and 195% and 454% for a combination of hemoglobin and cytokine stimulation in MNCs and PMNs, respectively (data not shown). The mean level of intracellular oxidants produced by the combination of cytokines and hemoglobin was greater than the sum of the mean for leukocytes treated separately with cytokines and hemoglobin. To compare the effect of steroids or NO synthase inhibitors within each experiment, all treatment results had the background fluorescence in the non-stimulated leukocytes subtracted and then compared, as a percentage, to the level of fluorescent DCF in leukocytes activated with a combination of cytokines and hemoglobin (FIGS. 15-18).

The effects of E₂, cortisol, P₄, or a combination of E₂ and P₄ on levels of intracellular oxidants in MNCs and PMNs after 1 or 5 hours of cytokine and hemoglobin activation are depicted in FIGS. 15-18. Only the highest 5 of the 10 concentrations are displayed. The lower 5 concentrations (1 to 300 nM), of each steroid had negligible effects on oxidant levels in activated leukocytes. As compared to leukocytes activated with cytokines and hemoglobin for 1 and 5 h, selective steroids had a concentration-dependent dampening effect on oxidant levels in MNCs and little effect on oxidants in PMNs, except a consistent, although modest trend for cortisol to reduce oxidants in PMNs. Of the three steroids tested, E₂ (10 μM) had the greatest dampening effect (65%) on oxidant production after 1 h, as compared to 20% for cortisol, and 0% for P₄. Combining P₄ (0.6 μM) with E₂ concentrations further reduced intracellular oxidants by 10-20% over values for E₂ alone, with a 75% reduction at the highest E₂ concentration. In contrast to E₂, cortisol reduced oxidant levels in MNCs to a greater extent after 5 h as compared to 1 h of stimulation.

Cell membrane damage in cytokine- and hemoglobin-activated MNCs and PMNs did not differ from unstimulated leukocytes, or any steroid or NOS inhibitor treatments (data not shown). These results support the idea that the treatment effects were not due to cell membrane damage allowing the DCF fluorescent probe to leak from the leukocyte. Overall, L-NMMA inhibited 80% of the cytokine- and hemoglobin-induced oxidant production in PMNs and 50% in MNCs. The specific inhibitor of inducible NO Synthase, L-NIL, had negligible effects on activated MNCs and PMNs. These results suggest that the predominant oxidant inhibited by the steroids is NO, probably derived from constitutive NOS. In only MNCs activated for 5 h, the supposedly inactive stereoisomer D-NMMA appeared to reduce intracellular oxidant levels.

Previous reports have shown that treatment with E₂, cortisol, or P₄ improves outcome in a variety of injury models. Recently, studies in several injury models have demonstrated that E₂ effects are receptor-mediated and, therefore, not attributable to E₂ antioxidant effect, and demonstrated a time-dependent affect on injury (0.5-3 h) with optimal treatment within 0.5 h of injury. In this study, pharmacological concentrations of E₂ markedly reduced intracellular levels of oxidants in MNCs, but not in PMNs, further supporting the concept that E₂ effects in MNCs were not due to its antioxidant properties.

Multiple studies have found E₂ receptors in/on MNCs, whereas, E₂ receptors were found on PMNs only after activation. Cortisol receptors in both MNCs and PMNs were approximately equal and present without activation. No reports of P₄ receptors in/on leukocytes were found. Interestingly, steroid effects on intracellular levels of oxidants appear to coincide with the reported levels of steroid receptors in/on MNCs and PMNs. For MNCs, pharmacological levels of E₂ (10 μM) reduced oxidants to the greatest extent, followed by cortisol, with P₄ having no discernible effects. Even though P₄ alone had no effect on intracellular oxidants, when combined with E₂ (3-10 μM), P₄ (0.6 μM) further decreased oxidants levels compared to E₂ alone. For PMNs, E₂ or P₄ had no effect on oxidants, while all cortisol concentrations showed a modest trend for reducing oxidant levels after 5 h. Thus, other non-steroidal treatments that dampen PMNs oxidant production may compliment E₂-demonstrated beneficial in variety of injury models. In contrast, physiological levels of E₂ (1-100 nM) have been reported to increase NO production by an E₂ receptor-mediated process in non-activated MNCs, establishing a major difference between activated and non-activated MNCs.

Similar to peripheral human monocytes, activated macrophages derived from lungs or a macrophage cell line (J774) treated with pharmacological levels of E₂ reduced NO production (Robert R, Spitzer J A: Effects of female hormones (17beta-estradiol and progesterone) on nitric oxide production by alveolar macrophages in rats. Nitric Oxide 1: 453-462, 1997; Hayshi T, Yamada K, Esaki T, Muto E, Chauhuri G, Iguchi A: Physiological concentrations of 17beta-estradiol inhibit the synthesis of nitric oxide synthase in macrophages via a receptor-mediated system. J Cardiovas Pharmacol 31:292-298, 1998). Nitric oxide, generated by cNOS or iNOS, has been demonstrated to act as an early proinflammatory mediator in both MNCs and PMNs by increasing inflammatory transcription factors (NF-κβ) and cytokine expression (TNFα and IL1β) that subsequently activate inflammatory cascades resulting in lung and liver injury. One of the mechanisms by which pharmacological levels of E₂ may reduce tissue injury is by inhibiting NO-induced cytokines and chemokines synthesis in monocytes and macrophages that amplify the early events in the inflammatory cascade. Since NO is permeant to cell membranes, and binds to sulfhydryl groups and heme moieties in regulatory proteins resulting a potential redox alteration in neighboring cells, E₂ may also dampen NO paracrine and toxic effects on neighboring cells.

The early dampening of adhesion and of the subsequent extravasation of leukocytes into injured tissues by E₂-mediated attenuation of inflammatory cytokines has been proposed as a pathway for E₂ salutary effects. This mechanism is consistent with the requirement of E₂ treatment within 0.5 h of injury for optimal beneficial effects. In contrast to treatment with NO synthase inhibitors that have been reported to increase certain types of injuries, E₂ reduces injury while concomitantly increasing endothelial NO synthase levels and perfusion of ischemic tissues. Results of this study suggest that E₂ may limit NO production in monocytic cells that initiate early inflammatory events. These attributes of E₂ may have unique therapeutic value in the treatment of the severely injured, as has already been demonstrated in a variety of injury models.

Higher E₂ and E₂ precursor (DHEA) levels have been linked to an improved outcome following injury. DHEA and T are converted to E₂ by aromatase, which its expression and activity in tissues is increased by inflammatory cytokines and glucocorticoids following trauma. Interestingly, concentrations of DHEA (0.2 uM) and conjugate DHEA sulfate (0.1-10 uM), the most abundant steroid in circulation, of young adults are sufficient, if converted, to generate pharmacological levels of E₂ in injured tissue. Combining cortisol with E₂ for the early treatment of severe injury may provide not only a modest dampening effect on oxidant production by PMNs and lower oxidants in MNCs for a longer period of time (up to 5 h), but also increase the aromatization of DHEA and of deleterious androgens to E₂ in injured tissues.

PMNs and MNCs isolated within 2 h from 6 healthy male donors were selected as a model to compare the effect of equimolar concentrations of E₂, cortisol, P₄, and the combination of E₂ and P₄ on intracellular oxidant production. The current “two hit” phenomenon or the multiple hit paradigm of severe inflammation, leading to, for example, systemic inflammatory response syndrome (SIRS) or multiple organ dysfunction syndrome (MODS), consists of an initial insult such as hemorrhage, which primes the immune system for a amplified response to a second or subsequent inflammatory mediator(s). To mirror the multiple inflammatory mediators released following severe trauma, we used a cocktail of human inflammatory cytokines (TNFα, IL1β and INFγ), previously shown to be released from injured tissues, in combination with human hemoglobin as the ‘second hit’. Free hemoglobin is found in blood following trauma and transfusions. Hemoglobin and its breakdown product, heme, are proinflammatory mediators that can further augment oxidant production in leukocytes primed by other activating molecules (29. We found in both MNCs and PMNs that the combined effect of the cytokine mix and hemoglobin on intracellular oxidants was greater than the oxidant levels induced by either cytokine mix or hemoglobin, suggesting that this ‘multiple hit’ would amplify the inflammatory cascade.

Although the present invention has been described in terms of a particular preferred embodiments, it is not limited to those embodiments. Alternative embodiments, examples, and modifications which would still be encompassed by the invention may be made by those skilled in the art, particularly in light of the foregoing teachings.

1. A method for determining the presence of one or more immune response mediators, comprising obtaining a solution containing polymorphonuclear neutrophils, exposing the neutrophils to a chromophore, allowing the chromophore to oxidize to form a luminescent compound, and measuring the level of visible or ultraviolet radiation.

2. The method of claim 1 wherein the immune response mediator is an inflammation mediators.

3. The method of claim 1 wherein the luminescent compound is a fluorescent compound.

4. The method of claim 1 wherein the level of radiation is an indicator of the amount of deleterious effect caused by the inflammation mediator.

5. The method of claim 1 wherein the level of radiation is an indicator of the amount of counter-agent to the inflammation mediator that may be appropriate to administer to the human or animal that produced the inflammation response.

6. The method of claim 1 wherein the solution containing polymorphonuclear neutrophils comprises a body fluid.

7. The method of claim 1 wherein the chromophore is any molecule, or group or groups in a molecule, which absorb visible or ultraviolet radiation.

8. The method of claim 8 wherein the groups include —CH═CH—, —N═N—, or —C(═O)—.

9. The method of claim 1 wherein the is a fluorescein compound.

10. The method of claim 9 wherein the fluorescein compound comprises a salt of acetic acid.

11. The method of claim 10 wherein the fluorescein compound is 2,7-dichlorodihydrofluorescein diacetate.

12. The method of claim 9 wherein the fluorescein compound is conjugated.

13. The method of claim 12 wherein the fluorescein compound is conjugated to hemoglobin or myoglobin.

14. The method of claim 1 wherein allowing the chromophore to oxidize comprises contacting the chromophore with intracellular oxidants formed as a function of the inflammation process.

15. The method of claim 1 wherein the method of measuring the level of radiation involves measuring the amount of any chromophore suitable for use with flow cytometry.

16. A method of treatment comprising determining the level of inflammation mediators present in a body fluid, and administering an agent counter to the inflammation mediator.

17. A method of treatment comprising the use of hemoglobin-conjugated fluorescein to evaluate endocytic processes that are indicators of inflammation mediation.

18. A method for screening or evaluating drugs that modulate one or more steps in the inflammation cascade comprising obtaining a solution containing polymorphonuclear neutrophils, exposing the neutrophils to a chromophore, allowing the chromophore to oxidize to form a luminescent compound, and measuring the level of visible or ultraviolet radiation.

19. A method of screening or evaluating various concentrations of drugs comprising obtaining a solution containing polymorphonuclear neutrophils, exposing the neutrophils to a chromophore, allowing the chromophore to oxidize to form a luminescent compound, and measuring the level of visible or ultraviolet radiation.

20. A method of screening or evaluating combination of drugs and/or combinations of concentrations comprising obtaining a solution containing polymorphonuclear neutrophils, exposing the neutrophils to a chromophore, allowing the chromophore to oxidize to form a luminescent compound, and measuring the level of visible or ultraviolet radiation.
